(19) United States
(12) Patent Application Publication
(10) Pub. No.: US 2025/0137007 A1
(43) Pub. Date: May 1, 2025

(54) TRANSGENIC SEEDS FOR INHIBITING VIRAL REPLICATION VIA ANTICOMMUNICABLE FORTIFICATION SYSTEM (ACF SYSTEM) WITH CONCOMITANT METHOD OF TREATMENT OR PREVENTION OF VIRAL DISEASE, AND PANDEMIC PREVENTION AND ELIMINATION

(71) Applicant: David Kotlyar, Sterling, VA (US)

(72) Inventor: David Kotlyar, Sterling, VA (US)

(21) Appl. No.: 18/834,978

(22) PCT Filed: Jan. 31, 2023
(86) PCT No.: PCT/US23/12031

§ 371 Date: Jul. 31, 2024

Related U.S. Application Data
(60) Provisional application No. 63/305,137, filed on Jan. 31, 2022.

(51) Int. Cl. C12N 15/82; A61K 31/4015; A61K 31/427; A61K 31/675; A61K 31/706; A61K 36/48; A61K 36/899; A61K 36/8998; A61P 31/14; C07K 14/42

(52) U.S. Cl. CPC C12N 15/8257; A61K 31/4015; A61K 31/427; A61K 31/675; A61K 31/706; A61K 36/48; A61K 36/899; A61K 36/8998; A61P 31/14; C07K 14/42

(57) ABSTRACT

Disclosed is an alternate method of prevention and treatment of diseases caused by viral pathogens of human being and animals including coronaviruses, influenza viruses, etc., by food supplementation or fortification of food with foodstuff derived from transgenic plants or parts thereof obtained from the presently disclosed transgenic plant seed. This method provides said transgenic plant seed, wherein the genome of said seed comprises an exogenous recombinant polynucleotide encoding a combination of polypeptides, which are carbohydrate binding proteins, particularly lectins that in combination as disclosed herein are combined in a manner to overcome potential toxicity caused by their transgenic expression and to lower the concentrations of each of the carbohydrate binding proteins when expressed as a part of the combination when compared to the individual transgenic expression of any of said carbohydrate binding proteins, and wherein said seed exhibit inhibition of viral replication and provide immunity against viral pathogens.

### CROSS REFERENCE TO RELATED APPLICATIONS

The current application claims benefit of the U.S. Provisional Patent Application 63/305,137 filed Jan. 31, 2022.

### FIELD OF THE INVENTION

The present invention generally relates to agriculture, food, transgenic plant seed and plants derived therefrom, and viral disease. More specifically, relates to a transgenic plant seed for inhibiting viral replication and providing immunity against viral pathogens and a method of treatment or prevention of viral disease involving foodstuff produced from plants and plant parts obtained from said transgenic plant seed.

### BACKGROUND OF THE INVENTION

Communicable diseases are illnesses caused by pathogenic biological agents, including viruses, bacteria, fungi, parasites, and protozoa. Such diseases spread among people through contact with contaminated surfaces, bodily fluids, or blood products, or through the air, insect bites, or consuming contaminated food and beverages. Although some communicable diseases can be treated or prevented by taking medication and vaccines, there has been an increase in awareness of adopting a healthy diet and food supplementation to aid in the prevention and reversal of these diseases. Nutrients, besides playing an important role in maintaining normal physiology of human's body and healthiness, are also required for enhancing the immunity of the body and can be effective against viral infections. They can present antiviral capacity either by entering into the defensive mechanism directly through interfering with the target viruses, or indirectly through activating the cells associated with the adaptive immune system.

The viral infections can be highly contagious and easily transmissible, which even can lead to a pandemic, like the recent COVID-19 outbreak, causing massive deaths worldwide. While, still the best practical way to prevent the transmission of viruses is to practice self-sanitation and follow social distancing principles, enhancing the individual's immunity through the consumption of proper foods containing balanced nutrients can have significant result against viral infections. Foods containing nutrients such as vitamins, minerals, fatty acids, various polysaccharides, and some non-nutrients (i.e., polyphenols) have shown therapeutic potential against the function of viruses.

Many of the viral infections are highly contagious and easily transmissible which can lead to massive health problems or even deaths worldwide. Some of the emerging viral infections are caused by viruses such as Measles, HIV, influenza, herpes simplex virus, dengue, Chikungunya, zika, hepatitis, etc. Few other viruses such as severe acute respiratory syndrome (SARS) and middle east respiratory syndrome (MERS), causing respiratory illness, have also emerged recently. The outbreak of Spanish flu, caused by the H1N1 influenza virus at the beginning of the 20th century has been one of the deadliest viruses in human history (CDC and WHO). The recent outbreak of COVID-19, caused by the novel coronavirus SARS-CoV-2, related to the respiratory syndrome, was declared as pandemic by the WHO in March 2020.

The COVID-19 pandemic, also known as the coronavirus pandemic, is an ongoing pandemic of coronavirus disease 2019 (COVID-19) caused by severe acute respiratory syndrome coronavirus 2 (SARS-CoV-2). Despite the wide range of antiviral drugs currently available to potentially be used in the process of finding an appropriate cure along with vaccines being made available to the public at large, the sudden emergence of novel viral strains, mutation-driven resistance of viruses against such developed antivirals and vaccines remains a major problem in the art and requires sustainable, easily available, and mass produced solutions that also inhibit or prohibit and target viral infections at the inception including curbing viral replication and/or viral entry into the individual hosts.

Great progress has been made over the past decades with respect to the application of biotechnology to generate nutritionally improved food crops. Biofortified staple crops such as rice, maize and wheat harboring essential micronutrients as well as new varieties of transgenic crops have the ability to combat chronic diseases. Accordingly, a transgenic crop that may be utilized as a food supplement, and that can employ widespread protection against viral pathogens including but not limited to viruses such as measles, HIV, influenza, herpes simplex virus, dengue, chikungunya, zika, hepatitis, etc. Few other viruses such as severe acute respiratory syndrome (SARS) and middle east respiratory syndrome (MERS), and the recent pandemic causing pathogen, SARS-CoV-2 is a need of the hour. The present invention is intended to meet these needs and provide a solution to the above highlighted problem.

### SUMMARY OF THE INVENTION

The present disclosure provides a transgenic plant seed comprising an exogenous recombinant polynucleotide encoding a combination of carbohydrate binding proteins for inhibiting viral replication and providing immunity against viral pathogens. Further, the present disclosure provides a method of treatment or prevention of viral disease comprising administering to an individual in need an effective amount of a foodstuff derived from plants and plant parts obtained from a transgenic plant seed comprising an exogenous recombinant polynucleotide encoding a combination of carbohydrate binding proteins for inhibiting viral replication and providing immunity against viral pathogens.

In an aspect of the present invention, a transgenic plant seed for inhibiting viral replication and providing immunity against viral pathogens is disclosed, wherein the genome of said seed comprises an exogenous recombinant polynucleotide encoding a combination of polypeptides, wherein said polypeptides are carbohydrate binding proteins, and wherein said seed exhibit inhibition of viral replication and provide immunity against viral pathogens.

In another aspect of the present invention, the transgenic plant seed for inhibiting viral replication and providing immunity against viral pathogens as disclosed herein, wherein the transgenic plant seed are sown to produce plants and plant parts that are mass produced with agricultural practices, horticulture practices, and in green house gardens, wherein said plant parts comprise grains, harvested seeds, and by-products, wherein said mass produced plants and plant parts are processed to produce foodstuff, wherein said foodstuff comprise bread, cereal, flour, baby food, snack food, pet food, dried soups, dry beverage mixes, and texturized vegetable proteins, wherein said by-products comprise bran, middlings, mill run, shorts, red dog, screenings, germ meal, and germ oil, and wherein said by-products produce animal feed and manures, and wherein said foodstuff exhibit inhibition of viral replication and provide immunity against viral pathogens.

In an aspect of the present invention, a recombinant polynucleotide encoding a combination of polypeptides is disclosed, wherein said polypeptides are carbohydrate binding proteins, wherein the carbohydrate binding proteins are encoded by the recombinant polynucleotide encoding the combination of polypeptides, wherein the recombinant polynucleotide encoding the combination of polypeptides is under the control of promoters which are selected from heterologous and homologous promoters, wherein the promoters lead to ubiquitous expression or a plant part specific targeted expression from the recombinant polynucleotide encoding the combination of polypeptides, wherein the promoters are functional in a plant cell and operably joined to encoding sequence of the recombinant polynucleotide encoding the combination of polypeptides, wherein the combination of polypeptides is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides, and wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

In an aspect of the present invention, a method for treating or preventing a viral disease is disclosed, comprising administering to an individual in need an effective amount of a foodstuff derived from plants and plant parts obtained from a transgenic plant seed, the method comprising the steps of:
(a) making a recombinant polynucleotide encoding a combination of polypeptides, wherein said polypeptides are carbohydrate binding proteins, wherein the carbohydrate binding proteins are encoded by the recombinant polynucleotide encoding the combination of polypeptides, wherein the recombinant polynucleotide encoding the combination of polypeptides is under the control of promoters which are selected from heterologous and homologous promoters, wherein the promoters lead to ubiquitous expression or a plant part specific targeted expression from the recombinant polynucleotide encoding the combination of polypeptides, wherein the promoters are functional in a plant cell and operably joined to encoding sequence of the recombinant polynucleotide encoding the combination of polypeptides;
(b) transforming plant cells by delivering the recombinant polynucleotide of step (a) and regenerating full fertile transformed plants from said cells in vitro, wherein the transformed plants express combination of polypeptides, wherein said polypeptides are carbohydrate binding proteins;
(c) growing said transformed plants to obtain a transgenic plant seed;
(d) sowing said transgenic plant seed to produce transgenic plants and plant parts that are mass produced with agricultural practices, horticulture practices, and in green house gardens; and
(e) processing said transgenic plants and plant parts to obtain foodstuff, wherein said foodstuff exhibit inhibition of viral replication and provide immunity against viral pathogens, wherein said plant parts comprise grains, harvested seeds, and by-products, wherein said mass produced plants and plant parts are processed to produce foodstuff, wherein said foodstuff comprise bread, cereal, flour, baby food, snack food, pet food, dried soups, dry beverage mixes, and texturized vegetable proteins, wherein said by-products comprise bran, middlings, mill run, shorts, red dog, screenings, germ meal, and germ oil, wherein said by-products produce animal feed and manures, and wherein the administering to an individual in need an effective amount of a foodstuff leads to reduction or elimination of viral reservoirs in the individual, wherein said viral reservoirs include viral deposits in gut and central nervous system (CNS) of the individual.

It is already an established fact that combination of carbohydrate binding proteins, particularly lectins will inhibit viral transcription. Lectins have potent antiviral properties, which has been attributed to their direct binding to viral envelope glycans, thus preventing viral cell entry. Thus, according to the present invention, foodstuff derived from genetically modified corn and wheat formed using a combination of lectins from a plurality of sources helps provide immunity against viral pathogens such as coronaviruses and influenza viruses. Further, mass producing such transgenic seeds and applying to food supply may help eradicate SARS-CoV-2 virus or influenza virus via population-based means.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention. In the drawings:

FIG. 1 provides a schematic representation of making a transgenic plant by first (a) making a recombinant polynucleotide transgenes construct comprising a combination of carbohydrate binding proteins (n—represents the number of different carbohydrate binding protein genes incorporated in the said construct at a time) for expression in plants, which is then used in (b) to transform plant cells using the Agrobacterium intermediary method for instance which leads to regeneration of transgenic plantlets that are then confirmed for the desired expression (at the desired concentrations) of the specific combination of carbohydrate binding proteins as empirically required and tested as an embodiment of the present disclosure, which then produce a transgenic plant seed as per the present disclosure.

FIG. 2 provides a schematic representation of carbohydrate binding proteins such as lectin-driven protection against viral pathogens where said combinations of lectins may be extracted and produced from the transgenic plant seed or the foodstuff derived therefrom and made as per the embodiments of the present disclosure as shown in FIG. 1 as an embodiment of the present disclosure and the underlying mechanisms that include (a) inhibiting nuclear import and there by replication of viral pathogens, (b) causing viral pathogen aggregation to be picked up for clearance via various host mechanisms, (c) binding and inhibiting host cell entry by binding to surface proteins on the viral pathogens to prevent their interaction with host cell receptors to prevent initial viral pathogen entry into host cells, where (a) to (c) mechanisms work in concert to neutralize the viral pathogens in the beginning thereby inhibiting or preventing reservoir formation of such viral pathogens that also curtails mutations and generation of other antiviral and vaccine resistant viral strains of such viral pathogens, and (d) provides in vivo protection in mice models that can be translated to hosts.

FIG. 3 provides a schematic representation of an in vitro toxicity and cell survival assay when cells are exposed to desired experimental concentrations of (a) the experimental combination of lectins of the present disclosure as compared with (b) the individual lectins forming the said combination as compared with (c) a control protein not known to provide any protection against viral pathogens as well as (d) non-treated Vero E6 cells to experimentally assay and determine toxicity and tolerance from such exposure and treatment, where the said combination of proteins or individual proteins may either be sourced directly from the original plant sources and combined in the desired concentrations in line with the concentrations of the same extracted from experimentally produced transgenic plants and transgenic plant seed in Example 1 as disclosed herein.

FIG. 4 provides a schematic representation of an in vitro assay to determine the antiviral activity of (a) the experimental combination of lectins as compared with (b) the individual lectins forming the said combination as compared with (c) a control protein not known to provide any protection against viral pathogens as well as (d) non-treated Vero E6 cells to experimentally assay and determine protection from viral pathogens such as influenza viruses, SARS-CoV-2, feline infectious peritonitis infection in Vero E6 cells, where the said combination of proteins or individual proteins may either be sourced directly from the original plant sources and combined in the desired concentrations in line with the concentrations of the same extracted from experimentally produced transgenic plants and transgenic plant seed in Example 1 as disclosed herein.

FIG. 5 provides a schematic representation of an in vivo antiviral activity of mice fed food pellets comprising foodstuff comprising desired experimental concentrations of (a) and (e) the experimental combination of lectins of the present disclosure as compared with (b) and (f) the individual lectins forming the said combination as compared with (c) and (g) a control protein not known to provide any protection against viral pathogens as well as (d) and (h) non-treated mice to experimentally assay and determine antiviral activity with such treatment and determine protection from viral pathogens and infections caused by pathogens such as influenza viruses, SARS-CoV-2, feline infectious peritonitis infection for mouse models identified as (I) direct exposure to viral pathogen based infection, and (II) contact-based infection transfer from an already infected mouse to unexposed mouse, where the said combination of proteins or individual proteins may either be sourced directly from the original plant sources and combined in the desired concentrations in line with the concentrations of the same extracted from experimentally produced transgenic plants and transgenic plant seed in Example 1 as disclosed herein.

### DETAILED DESCRIPTION OF THE INVENTION

The present invention may be understood more readily by reference to the following detailed description of the invention taken in connection with the accompanying drawing figures, which forms a part of this disclosure. It is to be understood that this invention is not limited to the specific devices, medicines, systems, conditions or parameters described and/or shown herein and that the terminology used herein is for the example only, and is not intended to be limiting of the claimed invention. Also, as used in the specification including the appended claims, the singular forms ‘a’, ‘an’, and ‘the’ include the plural, and references to a particular numerical value includes at least that particular value unless the content clearly directs otherwise. Ranges may be expressed herein as from ‘about’ or ‘approximately’ another particular value. When such a range is expressed, it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

The terms “comprising,” “having,” “including,” and “containing” are to be construed as open-ended terms (i.e., meaning “including, but not limited to,” and permit the presence of one or more features or components) unless otherwise noted. It should be understood that while various embodiments in the specification are presented using “comprising” language, under various circumstances, a related embodiment may also be described using “consisting of” or “consisting essentially of language.

As such, the terms “a” (or “an”), “one or more,” and “at least one” can be used interchangeably herein. Furthermore, “and/or” where used herein is to be taken as specific disclosure of each of the two specified features or components with or without the other. Further, unless specifically stated or obvious from context, as used herein, the term “or” is understood to be inclusive. Also, unless specifically stated or obvious from context, as used herein, the terms “a”, “an”, and “the” are understood to be singular or plural.

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5, etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH. In some embodiments, when the term “about” is used, it means the recited number plus or minus 10% of that recited number.

As used herein, the terms “transgenic plant seeds”, “transgenic plant”, “transgenic plant parts” relates to a plant or seed or plant cell whose genome has been altered by the incorporation of exogenous genetic material, e.g., by transformation as described herein. The term “transgenic plant” is used to refer to the plant produced from an original transformation event, or progeny from later generations or crosses of a plant so transformed, so long as the progeny contains the exogenous genetic material in its genome.

As used herein, the term “exogenous” is meant that a nucleic acid molecule, for example, a recombinant polynucleotide, originates from outside the plant into which it is introduced. An exogenous nucleic acid molecule may comprise naturally or non-naturally occurring polynucleotides, and may be derived from any organism, including the same or a different plant species than that into which it is introduced.

As used herein, the term “recombinant polynucleotide” refers to a polynucleotide having a genetically engineered modification introduced through manipulation via mutagenesis, restriction enzymes, and the like. Recombinant polynucleotides may comprise DNA segments obtained from different sources, or DNA segments obtained from the same source, but which have been manipulated to join DNA segments which do not naturally exist in the joined form. A recombinant polynucleotide may exist outside of the cell, for example as a PCR fragment, or integrated into a genome, such as a plant genome.

As used herein, the term “functional portion” of an encoding region for a polypeptide provided herein is a sufficient portion of the encoding region to provide the desired activity. Where expression of protein is desired, a functional portion will generally comprise the entire coding region for the polypeptide, although certain deletions, truncations, rearrangements and the like of the polypeptide may also maintain, or in some cases improve, the desired activity. One skilled in the art is aware of methods to screen for such desired modifications and such polypeptides are considered within the scope of the present invention. Where gene suppression methods are employed, smaller portions of the encoding region may be used to produce the desired effect.

As used herein, the term “control plant” is a plant used to compare against a transgenic plant grown from transgenic seed provided herein, to identify an enhanced phenotype in said transgenic plant. A suitable control plant may be a non-transgenic plant of the parental line used to generate a transgenic plant herein. A control plant may in some cases be a transgenic plant line that comprises an empty vector or marker gene, but does not contain the recombinant polynucleotide of the present invention that is expressed in the transgenic plant being evaluated. In general, a control plant is a plant of the same line or variety as the transgenic plant being tested.

As used herein, the term “expression” as used herein refers to transcription of DNA to produce RNA. The resulting RNA may be without limitation mRNA encoding a protein, antisense RNA that is complementary to an mRNA encoding a protein, or an RNA transcript comprising a combination of sense and antisense gene regions, such as for use in RNAi technology. “Encoding” as used herein further refers to production of encoded protein from mRNA.

As used herein, the terms “foodstuff”, “food”, “food supplement”, which are used interchangeably and mean and include bread, cereal, flour, baby food, snack food, pet food, dried soups, dry beverage mixes, and texturized vegetable proteins, as well as animal feed derived from by-products of transgenic plants obtained from the transgenic plant seed as disclosed herein.

As used herein the terms “anti-communicable fortification system (ACF) of foodstuffs/seeds” means and includes ACF comprising transgenic plant seed, plants and plant parts prepared and obtained from said transgenic plant seed, and foodstuffs produced and obtained therefrom, wherein said seed, plants, plant parts, and foodstuff exhibit inhibition of viral replication and provide immunity against viral pathogens.

As used herein the terms “anti-communicable fortification system (ACF) of agricultural space” means and includes ACF to protect agriculture from damaging pathogens and relates to ACF specifically for the agricultural feed market.

As used herein, the term “viral disease” means and includes viral infection, and the resulting symptoms and pathogenesis and manifestation of the various stages and symptoms of a viral disease caused by the exposure to a viral pathogen.

As used herein, the term “effective amount” means the amount of an agent or composition or foodstuff or food supplement as disclosed herein required to prevent or ameliorate/treat the symptoms of a disease relative to an untreated individual exposed to or could be exposed to a viral pathogen, in which case it provides or boosts immunity against such viral pathogen. The effective amount of the active compound(s) or composition used to practice the present invention for prevention or curative treatment of a disease varies depending upon the manner of administration, the age, body weight, and general health of the subject. Ultimately, the attending physician or dietician or veterinarian will decide the appropriate amount and dosage regimen. Such amount is referred to as an “effective” amount.

As used herein, the terms “prevent”, “preventing”, “prevention,” and the like refer to actions taken to decrease the chance of getting a disease or condition, particularly caused by viral pathogens as disclosed herein. It will be appreciated that, although not precluded, preventing a disease, or disorder or condition does not require that the disorder, condition or symptoms associated therewith are completely avoided and a milder form is encompassed within this definition. Thus, the prevention and the like could be complete or partial.

As used herein, the terms “treat”, “treating”, “treatment,” and the like refer to reducing or ameliorating a disease or disorder and/or symptoms associated therewith, particularly caused by viral pathogens as disclosed herein. It will be appreciated that, although not precluded, treating a disorder or condition does not require that the disorder, condition or symptoms associated therewith be completely eliminated. Thus, the treatment and the like could be complete or partial.

As use/d herein, the terms “slate”, “slate system”, “slate system for the anti-communicable fortification system (ACF) of foodstuffs/seeds” means and includes a premade construction in anticipation that additional transgenic sequences will be added to the ACF to create a dynamic and flexible system for future diseases and pandemics of unknown import and effect. Such a slate system will be employed to modify and add other polypeptide encoding sequences to boost protection for humans in addition and in combination to creating a systematic plan for ACF.

In any of the ranges described herein, the endpoints of the range are included in the range. However, the description also contemplates the same ranges in which the lower and/or the higher endpoint is excluded. Additional features and variations of the invention will be apparent to those skilled in tire art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

Unless defined otherwise, all technical and scientific terms used herein have the same meaning as commonly understood by one of ordinary skill in the art to which this disclosure is related. Units, prefixes, and symbols are denoted in their Systems International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. The entire document is intended to be viewed as a unified disclosure, and it should be understood that all combinations of features described herein are contemplated. All references cited herein are hereby incorporated by reference in their entireties.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any foodstuffs, transgenic plant seed, transgenic plants and plant parts thereof, recombinant polynucleotide, polypeptides, carbohydrate binding proteins, lectins, or methods provided herein can be combined with one or more of any of the other compositions and methods provided herein.

As discussed hereinabove, there remains a problem and need for alternate prevention and treatment strategies and mechanisms for communicable and easily transmissible diseases caused by viral pathogens such as coronaviruses, rhabdoviruses, influenza viruses, dengue viruses, severe acute respiratory syndrome coronaviruses (SARS-CoV), severe acute respiratory syndrome coronavirus 1 (SARS-CoV-1), severe acute respiratory syndrome coronavirus 2 (SARS-CoV-2), middle east respiratory syndrome coronaviruses (MERS-CoV), orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses, HCoV-OC43, HCoV-229E, HCoV-NL63, HCoV-HKU1, feline infectious peritonitis virus (FIPV), feline enteric coronavirus (FECV), rinderpest virus, foot-and-mouth disease virus (FMDV), cypoviruses (CPV), and reoviruses. The existing antivirals, vaccines, and other pharmacological methodologies are insufficient and even fail to address the need of public health and management of communicable viral diseases and prevention of spread of communicable viral diseases associated with the sudden emergence of novel viral strains, mutation-driven resistance of viruses against such developed antivirals and vaccines. The need of the hour is for an alternate means and method that provides sustainable, easily available, and mass-produced solutions that can inhibit or prohibit and target viral infections at the inception including curbing viral replication and/or viral entry into the individual hosts to stop and prevent viral disease as well as increase immunity against viral pathogens.

To address the abovementioned problems and need in the area of communicable viral diseases which may even develop into pandemics like the recent COVID-19 pandemic, the present disclosure provides an alternate solution in the form of foodstuff or food supplementation or fortification in the form of foodstuff derived from transgenic plants or parts thereof obtained from the disclosed transgenic plant seed as disclosed herein that have an ability to prevent, combat, and even treat acute and chronic viral diseases caused by aforementioned viral pathogens. Such foodstuff can be incorporated easily into the diet of people across the globe and can even be used to complement and substantiate the antivirals, vaccines and other pharmacological means and methods against such viral pathogens by specifically reducing or even eliminating reservoirs in individuals exposed or may be exposed to such pathogens, where the reservoirs often include the gut and central nervous system (CNS) of such individuals, which may be human beings or animals who consume foodstuff made and disclosed according to the present disclosure.

An objective of the present invention is to provide a set of proteins which may be used alone or in combination to produce transgenic corn and/or wheat, that can inhibit replication of viral pathogens such as SARS-CoV-2, influenza virus, etc. In other words, transgenic plants such as transgenic corn and transgenic wheat produced using the specific polypeptides as disclosed in the present invention may be mass produced and utilized for making fortification of food consumed by an individual, be it a human being or an animal, in form of foodstuff such as bread and other food products to impart widespread protection against the viral pathogens.

It is already an established fact in the art that combination of lectins will inhibit viral transcription. Lectins have potent antiviral properties, which has been attributed to their direct binding to viral envelope glycans, thus preventing viral cell entry. Efficacy of lectins has been shown in several research studies. The following Table 1 provides a few examples of prior studies and published work that have shown promise in inhibiting the viral pathogen, SARS-CoV-2 the cause behind the COVID-19 pandemic:

TABLE 1
Carbohydrate binding proteins known to block viral infections

| Carbohydrate binding protein | Publication |
|---|---|
| DLL-I from lablab beans | Liu, Yo-Min, et al. “A carbohydrate-binding protein |
| | from the edible Lablab beans effectively blocks the |
| | infections of influenza viruses and SARS-CoV-2.” Cell |
| | reports 32.6 (2020): 108016 |
| GlcNAc-specific agglutinins | Barre, Annick, et al. “Man-specific lectins from plants, |
| from tobacco | fungi, algae and cyanobacteria, as potential blockers for |
| | SARS-CoV, MERS-CoV and SARS-CoV-2 (COVID- |
| | 19) coronaviruses: Biomedical perspectives.” Cells 10.7 |
| | (2021): 1619 |
| mannose specific agglutinin | Martinez, Daniela, et al. “The use of lectins as tools to |
| from leek | combat SARS-CoV-2.” Current Pharmaceutical Design |
| | 27.41 (2021): 4212-4222 |
| mannose specific agglutinin | Martinez, Daniela, et al. “The use of lectins as tools to |
| from Galanthus | combat SARS-CoV-2.” Current Pharmaceutical Design |
| | 27.41 (2021): 4212-4222 |

It has also been shown that these lectins may inhibit influenza and may significantly reduce morbidity and mortality globally due to endemic influenza.

Thus, according to the present invention, genetically modified corn and wheat formed using a combination of lectins from a plurality of sources helps provide immunity against coronavirus. Further, mass producing such transgenic seeds and applying to food supply may help eradicate viral pathogens via population-based means.

It should also be noted that, transgenic plants and crops may be transformed using recombinant polynucleotide encoding a combination of the above-mentioned lectin proteins, using any in vivo method or a combination of established techniques and/or methods that are known to one of ordinary skill in the art, as long as the intents of the present invention are not altered. Already established techniques on plant cultures have allowed the rapid increase of raw material availability through the use of suitable regeneration and multiplication systems. The novel aspect to this invention is the specific combination of the preceding lectins mentioned. Accordingly, transgenic seeds produced combining the above-mentioned lectins may then be mass produced for applying to food supply. Such mass-produced seeds may be utilized for making bread and other staple food products, that can protect against various viral pathogens including SARS-CoV-2. In other words, the present invention provides a novel and safe food supplement, which may possibly help to reduce or even eliminate reservoirs such as gut and CNS (central nervous system) of users who consume food products made according to the above-mentioned method. They may also serve as an adjunctive role with pharmacology to help prevent and/or supplement treatment of viral diseases such as COVID-19 disease or endemic influenza and reduce and/or eliminate viral loads and reservoirs in the host thereby not even allowing an opportunity for the viral pathogen to replicate and/or mutate and produce resistant viral strains.

In one embodiment of the present invention, it discloses a transgenic plant seed for inhibiting viral replication and providing immunity against viral pathogens, wherein the genome of said seed comprises an exogenous recombinant polynucleotide encoding a combination of polypeptides, wherein said polypeptides are carbohydrate binding proteins, and wherein said seed exhibits inhibition of viral replication and provide immunity against viral pathogens.

In another embodiment of the transgenic plant seed as disclosed herein, wherein the polypeptides in the combination of polypeptides are carbohydrate binding proteins, and wherein the combination of polypeptides is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides.

In another embodiment of the transgenic plant seed as disclosed herein, wherein the combination of polypeptides is a combination of carbohydrate binding proteins which are encoded by the exogenous recombinant polynucleotide encoding the combination of polypeptides, wherein the exogenous recombinant polynucleotide encoding the combination of polypeptides is under the control of promoters which are selected from heterologous and homologous promoters, wherein the promoters lead to ubiquitous expression or a plant part specific targeted expression from the exogenous recombinant polynucleotide encoding the combination of polypeptides, and wherein the promoters are functional in a plant cell and operably joined to encoding sequence of the recombinant polynucleotide encoding the combination of polypeptides.

In another embodiment of the transgenic plant seed as disclosed herein, wherein the combination of polypeptides is a combination of carbohydrate binding proteins, wherein the carbohydrate binding proteins are combined in a manner to overcome potential toxicity caused by transgenic expression of the carbohydrate binding proteins in the transgenic plant seed or plants grown from said seed and to lower the concentrations of each of the carbohydrate binding proteins when expressed as a part of the combination of carbohydrate binding proteins in the combination when compared with transgenic expression of any one of the carbohydrate binding proteins in the transgenic plant seed or plants grown from said seed individually, and wherein the concentration of the each of the carbohydrate binding proteins in the combination is in an approximate range of between 1 milligram/milliliter or 1 mg/ml to 1 attogram/milliliter or 1 ag/ml, usually in a typical range of between 50 microgram/milliliter or 50 μg/ml to 1 picogram/milliliter or 1 pg/ml. Some extraordinarily potent proteins and/or peptide sequences may have activity down to 1-10 ag/mL or 1-10 attogram/mL (attomolar concentration).

In another embodiment of the transgenic plant seed as disclosed herein, wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

In another embodiment of the transgenic plant seed as disclosed herein, wherein the carbohydrate binding proteins are lectins obtained from a plurality of sources that comprise glucose/mannose lectin or Dolichos lablab lectin 1 (DLL-I) from Lablab purpureus or lablab beans, GlcNAc-specific agglutinins from Nicotiana sp. or tobacco, mannose-specific agglutinin from Allium sp. or leek, mannose-specific agglutinin from Galanthus sp., mannose-specific lectin from rhizomes of Ophiopogon japonicus, chitin-specific lectin from rhizome of Setcreasea purpurea, Serpula vermicularis lectin (SVL), mannose-specific agglutinin from Hippeastrum hybrid or amaryllis, mannose-specific agglutinin from Galanthus nivalis or snowdrop, mannose-specific agglutinin from Narcissus pseudonarcissus or daffodil, mannose-specific agglutinin from Lycoris radiata or red spider lily, mannose-specific agglutinin from Allium porrum or leek, mannose-specific agglutinin from Allium ursinum or ramsons, mannose-specific agglutinin from Allium sativum or garlic, mannose-specific agglutinin from Colocasia esculenta or taro, mannose-specific agglutinin from Cymbidium hybrid or Cymbidium orchid, mannose-specific agglutinin from Listera ovata or twayblade, mannose-specific agglutinin from Epipactis helleborine or broad-leaved helleborine, mannose-specific agglutinin from Tulipa hybrid or tulip, mannose-specific agglutinin from Morus nigra or black mulberry tree, GlcNAc-specific agglutinins from Phragmites australis or common reed, GlcNAc-specific agglutinins from Nicotiana tabacum or tobacco plant, (GlcNAc)ₙ-specific agglutinin from Urtica dioica or stinging nettle, (GlcNAc)ₙ-specific agglutinin from Hevea brasiliensis or rubber tree, GalNAc-specific agglutinin from Polygonatum multiflorum tetramer or solomon's seal, GalNAc-specific agglutinin from Bryonia dioica or white bryony, GalNAc-specific agglutinin from Glechoma hederacea or ground ivy, Gal-specific agglutinin from Morus nigra or black mulberry tree, Gal-specific agglutinin from Artocarpus integrifolia or jackfruit, Neu5Acα(2.6)Gal/GalNAc-specific agglutinin from Sambucus nigra or elderberry, Man/Glc-specific agglutinin from Cladastris lutea or yellow wood, Gal/GalNAc-specific agglutinin from Polygonatum multiflorum monomer or solomon's Seal, Gal/GalNAc-specific agglutinin from Viscum album or mistletoe, GalNAc (>Gal) specific agglutinin from Viscum album or mistletoe, GalNAcα(1,3)Gal>GalNAc>Gal-specific agglutinin from Iris hybrid or Iris, Man/GalNAc-specific agglutinin from Tulipa hybrid or tulip, Hippeastrum hybrid agglutinin (HHA), Galanthus nivalis agglutinin (GNA), Cymbidium sp. agglutinin (CA), Urtica dioica agglutinin (UDA), Scytovirin (SVN) isolated from cyanobacterium Scytonema varium, carbohydrate binding proteins isolated from the sea coral Gerardia savaglia (GSA), Griffithsin derived from a red alga Griffithsia sp., and Actinohivin derived from the actinomycete Longisporum albida.

In another embodiment of the transgenic plant seed as disclosed herein, wherein the viral pathogens comprise coronaviruses, rhabdoviruses, influenza viruses, dengue viruses, severe acute respiratory syndrome coronaviruses (SARS-CoV), severe acute respiratory syndrome coronavirus 1 (SARS-CoV-1), severe acute respiratory syndrome coronavirus 2 (SARS-CoV-2), middle east respiratory syndrome coronaviruses (MERS-CoV), orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses, HCoV-OC43, HCoV-229E, HCoV-NL63, HCoV-HKU1, feline infectious peritonitis virus (FIPV), feline enteric coronavirus (FECV), rinderpest virus, foot-and-mouth disease virus (FMDV), cypoviruses (CPV), and reoviruses.

In another embodiment of the transgenic plant seed as disclosed herein, wherein the transgenic plant seed produces plants and plant parts that exhibit inhibition of viral replication and provide immunity against viral pathogens, wherein the plants comprise corn, wheat, millets, rye, oats, barley, sorghum, rice, legumes, nuts, and tubers.

In another embodiment of the transgenic plant seed as disclosed herein, wherein the transgenic plant seed are sown to produce plants and plant parts that are mass produced with agricultural practices, horticulture practices, and in green house gardens, wherein said plant parts comprise grains, harvested seeds, and by-products, wherein said mass produced plants and plant parts are processed to produce foodstuff, wherein said foodstuff comprise bread, cereal, flour, baby food, snack food, pet food, dried soups, dry beverage mixes, and texturized vegetable proteins, wherein said by-products comprise bran, middlings, mill run, shorts, red dog, screenings, germ meal, and germ oil, and wherein said by-products produce animal feed and manures, and wherein said foodstuff exhibit inhibition of viral replication and provide immunity against viral pathogens.

An aspect of the present disclosure relates to a slate system for the anti-communicable fortification system (ACF) of foodstuffs/seeds as disclosed in the present disclosure, wherein other rational or artificial intelligence (AI) derived combinations of lectins, lectin peptide fragments, or AI derived novel sequences derived from lectin and other antiviral peptide data can be used to update the original transgenic seeds as necessary for future pandemic agents and all known pathogens of clinical concern. Accordingly, in another embodiment of the transgenic plant seed as disclosed herein, wherein the polypeptides in the combination of polypeptides are carbohydrate binding proteins, and the carbohydrate binding proteins include any other rational or artificial intelligence (AI) derived combinations of lectins, lectin peptide fragments, or AI derived novel sequences derived from lectin and other antiviral peptide, wherein said seed exhibit inhibition of viral replication and provide immunity against viral pathogens, and wherein the viral pathogen include all known and unknown pathogens of clinical concern. The present disclosure in this aspect and embodiment of the present invention thus addresses any future need of upgradation of the transgenic plant seed or need for new transgenic plant seed that can be used in case of any resistance towards any viral pathogen, or need for coverage of a new pathogen, a new “slate” of new rational combinations can be added to the original transgenic seeds based on the disclosure of the present invention.

In one embodiment of the present invention, it discloses a recombinant polynucleotide encoding a combination of polypeptides, wherein said polypeptides are carbohydrate binding proteins, wherein the carbohydrate binding proteins are encoded by the recombinant polynucleotide encoding the combination of polypeptides, wherein the recombinant polynucleotide encoding the combination of polypeptides is under the control of promoters which are selected from heterologous and homologous promoters, wherein the promoters lead to ubiquitous expression or a plant part specific targeted expression from the recombinant polynucleotide encoding the combination of polypeptides, wherein the promoters are functional in a plant cell and operably joined to encoding sequence of the recombinant polynucleotide encoding the combination of polypeptides, wherein the combination of polypeptides is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides, and wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

In another embodiment of the recombinant polynucleotide encoding a combination of polypeptides as disclosed herein, wherein the carbohydrate binding proteins are lectins obtained from a plurality of sources that comprise glucose/mannose lectin or Dolichos lablab lectin 1 (DLL-I) from Lablab purpureus or lablab beans, GlcNAc-specific agglutinins from Nicotiana sp. or tobacco, mannose-specific agglutinin from Allium sp. or leek, mannose-specific agglutinin from Galanthus sp., mannose-specific lectin from rhizomes of Ophiopogon japonicus, chitin-specific lectin from rhizome of Setcreasea purpurea, Serpula vermicularis lectin (SVL), mannose-specific agglutinin from Hippeastrum hybrid or amaryllis, mannose-specific agglutinin from Galanthus nivalis or snowdrop, mannose-specific agglutinin from Narcissus pseudonarcissus or daffodil, mannose-specific agglutinin from Lycoris radiata or red spider lily, mannose-specific agglutinin from Allium porrum or leek, mannose-specific agglutinin from Allium ursinum or ramsons, mannose-specific agglutinin from Allium sativum or garlic, mannose-specific agglutinin from Colocasia esculenta or taro, mannose-specific agglutinin from Cymbidium hybrid or Cymbidium orchid, mannose-specific agglutinin from Listera ovata or twayblade, mannose-specific agglutinin from Epipactis helleborine or broad-leaved helleborine, mannose-specific agglutinin from Tulipa hybrid or tulip, mannose-specific agglutinin from Morus nigra or black mulberry tree, GlcNAc-specific agglutinins from Phragmites australis or common reed, GlcNAc-specific agglutinins from Nicotiana tabacum or tobacco plant, (GlcNAc)ₙ-specific agglutinin from Urtica dioica or stinging nettle, (GlcNAc)ₙ-specific agglutinin from Hevea brasiliensis or rubber tree, GalNAc-specific agglutinin from Polygonatum multiflorum tetramer or solomon's seal, GalNAc-specific agglutinin from Bryonia dioica or white bryony, GalNAc-specific agglutinin from Glechoma hederacea or ground ivy, Gal-specific agglutinin from Morus nigra or black mulberry tree, Gal-specific agglutinin from Artocarpus integrifolia or jackfruit, Neu5Acα(2.6)Gal/GalNAc-specific agglutinin from Sambucus nigra or elderberry, Man/Glc-specific agglutinin from Cladastris lutea or yellow wood, Gal/GalNAc-specific agglutinin from Polygonatum multiflorum monomer or solomon's Seal, Gal/GalNAc-specific agglutinin from Viscum album or mistletoe, GalNAc (>Gal) specific agglutinin from Viscum album or mistletoe, GalNAcα(1,3)Gal>GalNAc>Gal-specific agglutinin from Iris hybrid or Iris, Man/GalNAc-specific agglutinin from Tulipa hybrid or tulip, Hippeastrum hybrid agglutinin (HHA), Galanthus nivalis agglutinin (GNA), Cymbidium sp. agglutinin (CA), Urtica dioica agglutinin (UDA), Scytovirin (SVN) isolated from cyanobacterium Scytonema varium, carbohydrate binding proteins isolated from the sea coral Gerardia savaglia (GSA), Griffithsin derived from a red alga Griffithsia sp., and Actinohivin derived from the actinomycete Longisporum albida.

In another embodiment of the recombinant polynucleotide encoding a combination of polypeptides as disclosed herein, wherein the polypeptides in the combination of polypeptides are carbohydrate binding proteins, and the carbohydrate binding proteins include any other rational or artificial intelligence (AI) derived combinations of lectins, lectin peptide fragments, or AI derived novel sequences derived from lectin and other antiviral peptide, wherein said seed exhibit inhibition of viral replication and provide immunity against viral pathogens, and wherein the viral pathogen include all known and unknown pathogens of clinical concern.

In one embodiment of the present invention, it discloses a method for treating or preventing a viral disease, comprising administering to an individual in need an effective amount of a foodstuff derived from plants and plant parts obtained from a transgenic plant seed, the method comprising the steps of: (a) making a recombinant polynucleotide encoding a combination of polypeptides, wherein said polypeptides are carbohydrate binding proteins, wherein the carbohydrate binding proteins are encoded by the recombinant polynucleotide encoding the combination of polypeptides, wherein the recombinant polynucleotide encoding the combination of polypeptides is under the control of promoters which are selected from heterologous and homologous promoters, wherein the promoters lead to ubiquitous expression or a plant part specific targeted expression from the recombinant polynucleotide encoding the combination of polypeptides, wherein the promoters are functional in a plant cell and operably joined to encoding sequence of the recombinant polynucleotide encoding the combination of polypeptides; (b) transforming plant cells by delivering the recombinant polynucleotide of step (a) and regenerating full fertile transformed plants from said cells in vitro, wherein the transformed plants express combination of polypeptides, wherein said polypeptides are carbohydrate binding proteins; (c) growing said transformed plants to obtain a transgenic plant seed; (d) sowing said transgenic plant seed to produce transgenic plants and plant parts that are mass produced with agricultural practices, horticulture practices, and in green house gardens; and (e) processing said transgenic plants and plant parts to obtain foodstuff, wherein said foodstuff exhibit inhibition of viral replication and provide immunity against viral pathogens, wherein said plant parts comprise grains, harvested seeds, and by-products, wherein said mass produced plants and plant parts are processed to produce foodstuff, wherein said foodstuff comprise bread, cereal, flour, baby food, snack food, pet food, dried soups, dry beverage mixes, and texturized vegetable proteins, wherein said by-products comprise bran, middlings, mill run, shorts, red dog, screenings, germ meal, and germ oil, wherein said by-products produce animal feed and manures, and wherein the administering to an individual in need an effective amount of a foodstuff leads to reduction or elimination of viral reservoirs in the individual, wherein said viral reservoirs include viral deposits in gut and central nervous system (CNS) of the individual.

In another embodiment of the method for treating or preventing a viral disease as disclosed herein, wherein the combination of polypeptides is a combination of carbohydrate binding proteins in step (a) and is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides.

In another embodiment of the method for treating or preventing a viral disease as disclosed herein, wherein the combination of polypeptides is a combination of carbohydrate binding proteins in step (a) and the carbohydrate binding proteins are combined in a manner to overcome potential toxicity caused by transgenic expression of the carbohydrate binding proteins in the transgenic plant seed or plants grown from said seed and to lower the concentrations of each of the carbohydrate binding proteins when expressed as a part of the combination of carbohydrate binding proteins in the combination when compared with transgenic expression of any one of the carbohydrate binding proteins in the transgenic plant seed or plants grown from said seed individually, and wherein the concentration of the each of the carbohydrate binding proteins in the combination is in an approximate range of between 1 milligram/milliliter or 1 mg/ml to 1 attogram/milliliter or 1 ag/ml, usually in a typical range of between 50 microgram/milliliter or 50 μg/ml to 1 picogram/milliliter or 1 pg/ml. Some extraordinarily potent proteins and/or peptide sequences may have activity down to 1-10 ag/mL or 1-10 attogram/mL (attomolar concentration).

In another embodiment of the method for treating or preventing a viral disease as disclosed herein, wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

In another embodiment of the method for treating or preventing a viral disease as disclosed herein, wherein the carbohydrate binding proteins are lectins obtained from a plurality of sources that comprise glucose/mannose lectin or Dolichos lablab lectin 1 (DLL-I) from Lablab purpureus or lablab beans, GlcNAc-specific agglutinins from Nicotiana sp. or tobacco, mannose-specific agglutinin from Allium sp. or leek, mannose-specific agglutinin from Galanthus sp., mannose-specific lectin from rhizomes of Ophiopogon japonicus, chitin-specific lectin from rhizome of Setcreasea purpurea, Serpula vermicularis lectin (SVL), mannose-specific agglutinin from Hippeastrum hybrid or amaryllis, mannose-specific agglutinin from Galanthus nivalis or snowdrop, mannose-specific agglutinin from Narcissus pseudonarcissus or daffodil, mannose-specific agglutinin from Lycoris radiata or red spider lily, mannose-specific agglutinin from Allium porrum or leek, mannose-specific agglutinin from Allium ursinum or ramsons, mannose-specific agglutinin from Allium sativum or garlic, mannose-specific agglutinin from Colocasia esculenta or taro, mannose-specific agglutinin from Cymbidium hybrid or Cymbidium orchid, mannose-specific agglutinin from Listera ovata or twayblade, mannose-specific agglutinin from Epipactis helleborine or broad-leaved helleborine, mannose-specific agglutinin from Tulipa hybrid or tulip, mannose-specific agglutinin from Morus nigra or black mulberry tree, GlcNAc-specific agglutinins from Phragmites australis or common reed, GlcNAc-specific agglutinins from Nicotiana tabacum or tobacco plant, (GlcNAc)ₙ-specific agglutinin from Urtica dioica or stinging nettle, (GlcNAc)ₙ-specific agglutinin from Hevea brasiliensis or rubber tree, GalNAc-specific agglutinin from Polygonatum multiflorum tetramer or solomon's seal, GalNAc-specific agglutinin from Bryonia dioica or white bryony, GalNAc-specific agglutinin from Glechoma hederacea or ground ivy, Gal-specific agglutinin from Morus nigra or black mulberry tree, Gal-specific agglutinin from Artocarpus integrifolia or jackfruit, Neu5Acα(2.6)Gal/GalNAc-specific agglutinin from Sambucus nigra or elderberry, Man/Glc-specific agglutinin from Cladastris lutea or yellow wood, Gal/GalNAc-specific agglutinin from Polygonatum multiflorum monomer or solomon's Seal, Gal/GalNAc-specific agglutinin from Viscum album or mistletoe, GalNAc (>Gal) specific agglutinin from Viscum album or mistletoe, GalNAcα(1,3)Gal>GalNAc>Gal-specific agglutinin from Iris hybrid or Iris, Man/GalNAc-specific agglutinin from Tulipa hybrid or tulip, Hippeastrum hybrid agglutinin (HHA), Galanthus nivalis agglutinin (GNA), Cymbidium sp. agglutinin (CA), Urtica dioica agglutinin (UDA), Scytovirin (SVN) isolated from cyanobacterium Scytonema varium, carbohydrate binding proteins isolated from the sea coral Gerardia savaglia (GSA), Griffithsin derived from a red alga Griffithsia sp., and Actinohivin derived from the actinomycete Longisporum albida.

In another embodiment of the method for treating or preventing a viral disease as disclosed herein, wherein the viral pathogens comprise coronaviruses, rhabdoviruses, influenza viruses, dengue viruses, severe acute respiratory syndrome coronaviruses (SARS-CoV), severe acute respiratory syndrome coronavirus 1 (SARS-CoV-1), severe acute respiratory syndrome coronavirus 2 (SARS-CoV-2), Middle East respiratory syndrome coronaviruses (MERS-CoV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses, HCoV-OC43, HCoV-229E, HCoV-NL63, HCoV-HKU1, feline infectious peritonitis virus (FIPV), feline enteric coronavirus (FECV), rinderpest virus, foot-and-mouth disease virus (FMDV), cypoviruses (CPV), and reoviruses.

In another embodiment of the method for treating or preventing a viral disease as disclosed herein, wherein the transformed plants in step (c) and consequent transgenic plants in step (e) comprise corn, wheat, millets, rye, oats, barley, sorghum, rice, legumes, nuts, and tubers.

In another embodiment of the method for treating or preventing a viral disease as disclosed herein, wherein the effective amount of a foodstuff derived from plants and plant parts obtained from a transgenic plant seed is administered along with one or more antivirals, wherein antivirals comprise Remdesivir, Nirmatrelvir with Ritonavir (Paxlovid), and Molnupiravir.

In another embodiment of the method for treating or preventing a viral disease as disclosed herein, wherein the individual comprises human beings, domesticated animals, farm animals, zoo animals, agricultural beasts, and wild animals.

In one embodiment of the present invention, it discloses a transgenic plant seed for inhibiting viral replication and providing immunity against viral pathogens for use in foodstuff derived from plants and plant parts obtained from said transgenic plant seed, wherein the transgenic plant seed produces plants and plant parts that exhibit inhibition of viral replication and provide immunity against viral pathogens, wherein the plants comprise corn, wheat, millets, rye, oats, barley, sorghum, rice, legumes, nuts, and tubers, wherein the transgenic plant seed are sown to produce plants and plant parts that are mass produced with agricultural practices, horticulture practices, and in green house gardens, wherein said plant parts comprise grains, harvested seeds, and by-products, wherein said mass produced plants and plant parts are processed to produce foodstuff, wherein said foodstuff comprise bread, cereal, flour, baby food, snack food, pet food, dried soups, dry beverage mixes, and texturized vegetable proteins, wherein said by-products comprise bran, middlings, mill run, shorts, red dog, screenings, germ meal, and germ oil, wherein said by-products produce animal feed and manures, and wherein said foodstuff exhibit inhibition of viral replication and provide immunity against viral pathogens.

In another embodiment of the transgenic plant seed for inhibiting viral replication and providing immunity against viral pathogens for use in foodstuff derived from plants and plant parts obtained from said transgenic plant seed as disclosed herein, wherein the genome of said seed comprises an exogenous recombinant polynucleotide encoding a combination of polypeptides, wherein said polypeptides are carbohydrate binding proteins, and wherein said seed exhibit inhibition of viral replication and provide immunity against viral pathogens.

In another embodiment of the transgenic plant seed for inhibiting viral replication and providing immunity against viral pathogens for use in foodstuff derived from plants and plant parts obtained from said transgenic plant seed as disclosed herein, wherein the polypeptides in the combination of polypeptides are carbohydrate binding proteins, and wherein the combination of polypeptides is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides.

In another embodiment of the transgenic plant seed for inhibiting viral replication and providing immunity against viral pathogens for use in foodstuff derived from plants and plant parts obtained from said transgenic plant seed as disclosed herein, wherein the combination of polypeptides is a combination of carbohydrate binding proteins which are encoded by the exogenous recombinant polynucleotide encoding the combination of polypeptides, wherein the exogenous recombinant polynucleotide encoding the combination of polypeptides is under the control of promoters which are selected from heterologous and homologous promoters, wherein the promoters lead to ubiquitous expression or a plant part specific targeted expression from the exogenous recombinant polynucleotide encoding the combination of polypeptides, and wherein the promoters are functional in a plant cell and operably joined to encoding sequence of the recombinant polynucleotide encoding the combination of polypeptides.

In another embodiment of the transgenic plant seed for inhibiting viral replication and providing immunity against viral pathogens for use in foodstuff derived from plants and plant parts obtained from said transgenic plant seed as disclosed herein, wherein the combination of polypeptides is a combination of carbohydrate binding proteins, wherein the carbohydrate binding proteins are combined in a manner to overcome potential toxicity caused by transgenic expression of the carbohydrate binding proteins in the transgenic plant seed or plants grown from said seed and to lower the concentrations of each of the carbohydrate binding proteins when expressed as a part of the combination of carbohydrate binding proteins in the combination when compared with transgenic expression of any one of the carbohydrate binding proteins in the transgenic plant seed or plants grown from said seed individually, and wherein the concentration of the each of the carbohydrate binding proteins in the combination is in an approximate range of between 1 milligram/milliliter or 1 mg/ml to 1 attogram/milliliter or 1 ag/ml, usually in a typical range of between 50 microgram/milliliter or 50 μg/ml to 1 picogram/milliliter or 1 pg/ml. Some extraordinarily potent proteins and/or peptide sequences may have activity down to 1-10 ag/mL or 1-10 attogram/mL (attomolar concentration).

In another embodiment of the transgenic plant seed for inhibiting viral replication and providing immunity against viral pathogens for use in foodstuff derived from plants and plant parts obtained from said transgenic plant seed as disclosed herein, wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

In another embodiment of the transgenic plant seed for inhibiting viral replication and providing immunity against viral pathogens for use in foodstuff derived from plants and plant parts obtained from said transgenic plant seed as disclosed herein, wherein the carbohydrate binding proteins are lectins obtained from a plurality of sources that comprise glucose/mannose lectin or Dolichos lablab lectin 1 (DLL-I) from Lablab purpureus or lablab beans, GlcNAc-specific agglutinins from Nicotiana sp. or tobacco, mannose-specific agglutinin from Allium sp. or leek, mannose-specific agglutinin from Galanthus sp., mannose-specific lectin from rhizomes of Ophiopogon japonicus, chitin-specific lectin from rhizome of Setcreasea purpurea, Serpula vermicularis lectin (SVL), mannose-specific agglutinin from Hippeastrum hybrid or amaryllis, mannose-specific agglutinin from Galanthus nivalis or snowdrop, mannose-specific agglutinin from Narcissus pseudonarcissus or daffodil, mannose-specific agglutinin from Lycoris radiata or red spider lily, mannose-specific agglutinin from Allium porrum or leek, mannose-specific agglutinin from Allium ursinum or ramsons, mannose-specific agglutinin from Allium sativum or garlic, mannose-specific agglutinin from Colocasia esculenta or taro, mannose-specific agglutinin from Cymbidium hybrid or Cymbidium orchid, mannose-specific agglutinin from Listera ovata or twayblade, mannose-specific agglutinin from Epipactis helleborine or broad-leaved helleborine, mannose-specific agglutinin from Tulipa hybrid or tulip, mannose-specific agglutinin from Morus nigra or black mulberry tree, GlcNAc-specific agglutinins from Phragmites australis or common reed, GlcNAc-specific agglutinins from Nicotiana tabacum or tobacco plant, (GlcNAc)ₙ-specific agglutinin from Urtica dioica or stinging nettle, (GlcNAc)ₙ-specific agglutinin from Hevea brasiliensis or rubber tree, GalNAc-specific agglutinin from Polygonatum multiflorum tetramer or solomon's seal, GalNAc-specific agglutinin from Bryonia dioica or white bryony, GalNAc-specific agglutinin from Glechoma hederacea or ground ivy, Gal-specific agglutinin from Morus nigra or black mulberry tree, Gal-specific agglutinin from Artocarpus integrifolia or jackfruit, Neu5Acα(2.6)Gal/GalNAc-specific agglutinin from Sambucus nigra or elderberry, Man/Glc-specific agglutinin from Cladastris lutea or yellow wood, Gal/GalNAc-specific agglutinin from Polygonatum multiflorum monomer or solomon's Seal, Gal/GalNAc-specific agglutinin from Viscum album or mistletoe, GalNAc (>Gal) specific agglutinin from Viscum album or mistletoe, GalNAcα(1,3)Gal>GalNAc>Gal-specific agglutinin from Iris hybrid or Iris, Man/GalNAc-specific agglutinin from Tulipa hybrid or tulip, Hippeastrum hybrid agglutinin (HHA), Galanthus nivalis agglutinin (GNA), Cymbidium sp. agglutinin (CA), Urtica dioica agglutinin (UDA), Scytovirin (SVN) isolated from cyanobacterium Scytonema varium, carbohydrate binding proteins isolated from the sea coral Gerardia savaglia (GSA), Griffithsin derived from a red alga Griffithsia sp., and Actinohivin derived from the actinomycete Longisporum albida.

In another embodiment of the transgenic plant seed for inhibiting viral replication and providing immunity against viral pathogens for use in foodstuff derived from plants and plant parts obtained from said transgenic plant seed as disclosed herein, wherein the viral pathogens comprise coronaviruses, rhabdoviruses, influenza viruses, dengue viruses, severe acute respiratory syndrome coronaviruses (SARS-CoV), severe acute respiratory syndrome coronavirus 1 (SARS-CoV-1), severe acute respiratory syndrome coronavirus 2 (SARS-CoV-2), middle east respiratory syndrome coronaviruses (MERS-CoV), orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses, HCoV-OC43, HCoV-229E, HCoV-NL63, HCoV-HKU1, feline infectious peritonitis virus (FIPV), feline enteric coronavirus (FECV), rinderpest virus, foot-and-mouth disease virus (FMDV), cypoviruses (CPV), and reoviruses.

In another embodiment of the transgenic plant seed for inhibiting viral replication and providing immunity against viral pathogens for use in foodstuff derived from plants and plant parts obtained from said transgenic plant seed as disclosed herein, wherein the foodstuff is used for treating or preventing a viral disease, comprising administering to an individual in need an effective amount of said foodstuff, wherein the administering to an individual in need an effective amount of a foodstuff leads to reduction or elimination of viral reservoirs in the individual, wherein said viral reservoirs include viral deposits in gut and central nervous system (CNS) of the individual, wherein the effective amount of a foodstuff derived from plants and plant parts obtained from a transgenic plant seed is administered along with one or more antivirals, wherein antivirals comprise Remdesivir, Nirmatrelvir with Ritonavir (Paxlovid), and Molnupiravir, and wherein the individual comprises human beings, domesticated animals, farm animals, zoo animals, agricultural beasts, and wild animals.

The invention will be further explained by the following Examples, which are intended to purely exemplary of the invention, and should not be considered as limiting the invention in any way.

### Example 1

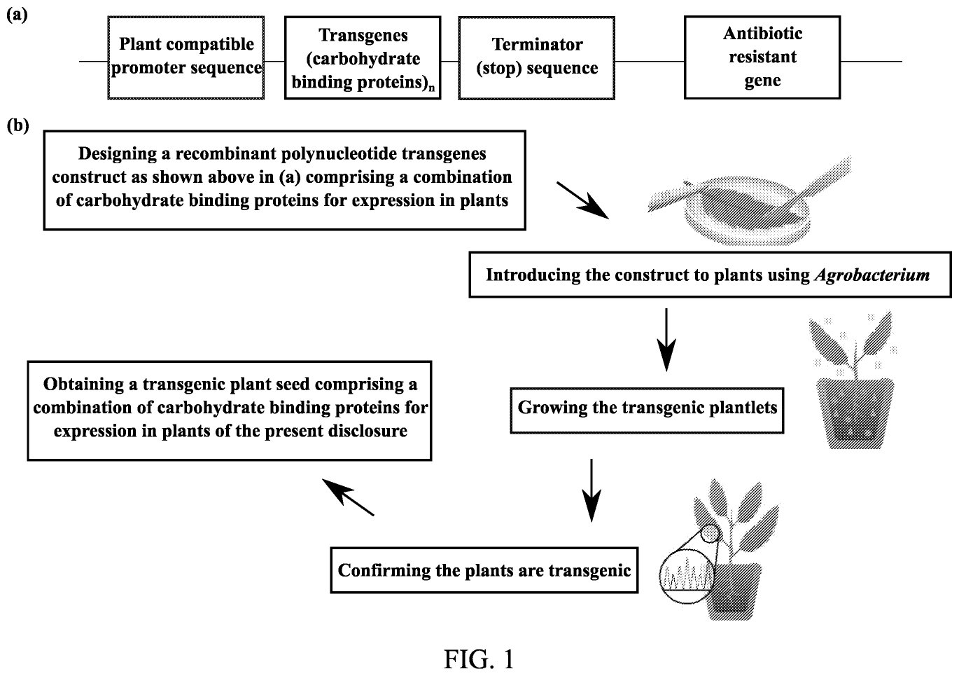

Preparation of one of the transgenic plant and transgenic plant seed: An experimental preparation of a transgenic plant seed from a transgenic plant will be carried out by first producing a recombinant polynucleotide encoding a combination of carbohydrate binding proteins like lectins, preferably, a combination of the lectins are chosen in various combinations from the Table 2 hereinbelow, wherein the recombinant polynucleotide encoding said combination is under the control of promoters which are selected from heterologous and homologous promoters, wherein the promoters lead to ubiquitous expression or a plant part specific targeted expression from the recombinant polynucleotide encoding the combination of lectins, wherein the promoters are functional in a plant cell and operably joined to encoding sequence of the recombinant polynucleotide encoding the aforementioned combination of lectins. One such experimental procedure that will be employed is schematically represented in FIG. 1(a).

TABLE 2
Plant-derived lectins known and being tested herein for their antiviral activity
(refer to: E. Keyaerts et al. Antiviral Research 75 (2007) 179-187)

| | | | | |
|---|---|---|---|---|
| Lectin | Plant species | Common name | Taxonomy | Reference |

Man-specific agglutinins

| | | | | |
|---|---|---|---|---|
| HHA | Hippeastrum hybrid | Amaryllis | Monocot, | Van Damme et |
| | | | Amaryllidaceae | al. (2002) |
| GNA | Galanthus nivalis | Snowdrop | Monocot, | Van Damme et |
| | | | Amaryllidaceae | al. (1998) |
| NPA | Narcissus | Daffodil | Monocot, | Van Damme et |
| | pseudonarcissus | | Amaryllidaceae | al. (1998) |
| LRA | Lycoris radiata | Red spider lily | Monocot, | Van Damme et |
| | | | Amaryllidaceae | al. (1998) |
| APA | Allium porrum | Leek | Monocot, | Van Damme et |
| | | | Alliaceae | al. (1998) |
| AUA | Allium ursinum | Ramsons | Monocot, | Van Damme et |
| | | | Alliaceae | al. (1998) |
| ASA | Allium sativum | Garlic | Monocot, | Van Damme et |
| | | | Alliaceae | al. (1998) |
| ASA I | Allium sativum | Garlic | Monocot, | Van Damme et |
| | | | Alliaceae | al. (1998) |
| Col O | Colocasia esculenta | Taro | Monocot, | Van Damme et |
| | | | Araceae | al. (1998) |
| CA | Cymbidium hybrid | Cymbidium | Monocot, | Van Damme et |
| | | orchid | Orchidaceae | al. (1998) |
| LOA | Listera ovata | Twayblade | Monocot, | Van Damme et |
| | | | Orchidaceae | al. (1998) |
| EHA | Epipactis | Broad-leaved | Monocot, | Van Damme et |
| | helleborine | helleborine | Orchidaceae | al. (1998) |
| TL M I | Tulipa hybrid | Tulip | Monocot, | Van Damme et |
| | | | Liliaceae | al. (1998) |
| Morniga M | Morus Nigra | Black mulberry | Dicotyl, | Van Damme et |
| II | | tree | Moraceae | al. (2002) |

GlcNAc-specific agglutinins

| | | | | |
|---|---|---|---|---|
| PallGlcNac | Phragmites australis | Common reed | Monocot, | Van Damme et |
| | | | Gramineae | al. (1998) |
| Nictaba | Nicotiana tabacum | Tobacco plant | Dicot, | Chen et al. |
| | | | Solanaceae | (2002) |

(GlcNAc)n-specific agglutinins

| | | | | |
|---|---|---|---|---|
| UDA | Urtica dioica | Stinging nettle | Dicot, | Van Damme et |
| | | | Urticaceae c | al. (1998) |
| Heveine | Hevea brasiliensis | Rubber tree | Dicot, | Van Damme et |
| | | | Euphorbiaceae | al. (1998) |

GalNAc-specific agglutinins

| | | | | |
|---|---|---|---|---|
| PMRIP t | Polygonatum | Solomon's seal | Monocot, | Van Damme et |
| | multiflorum tetramer | | Liliaceae | al. (1998) |
| BDA | Bryonia dioica | White bryony | Dicot, | Van Damme et |
| | | | Curcurbitaceae | al. (1998) |
| Glechoma | Glechoma | Ground ivy | Dicot, | Wang et al. |
| | hederacea | | Lamiaceae | (2003) |

Gal-specific agglutinins

| | | | | |
|---|---|---|---|---|
| Morniga G | Morus Nigra | Black mulberry | Dicot, | Van Damme et |
| II | | tree | Moraceae | al. (2002) |
| Jacalin | Artocarpus | Jackfruit | Dicot, | Van Damme et |
| | integrifolia | | Moraceae | al. (1998) |

Neu5Acα(2.6)Gal/GalNAc-specific agglutinins

| | | | | |
|---|---|---|---|---|
| SNA I | Sambucus nigra | Elderberry | Dicot, | Van Damme et |
| | | | Sambucaceae | al. (1998) |

Man/Glc-specific agglutinins

| | | | | |
|---|---|---|---|---|
| Cladistris | Cladastris lutea | Yellow wood | Dicot, Fabaceae | Van Damme et |
| | | | | al. (1998) |

Gal/GalNAc specific agglutinins

| | | | | |
|---|---|---|---|---|
| PMRIP m | Polygonatum | Solomon's Seal | Monocot, | Van Damme et |
| | multiflorum | | Liliaceae | al. (1998) |
| | monomer | | | |
| ML II | Viscum album | Mistletoe | Dicotyledoneae, | Van Damme et |
| | | | Viscaceae | al. (1998) |

GalNAc (>Gal) specific agglutinins

| | | | | |
|---|---|---|---|---|
| ML III | Viscum album | Mistletoe | Dicotyl, | Van Damme et |
| | | | Viscaceae | al. (1998) |

GalNAcα(1,3)Gal > GalNAc > Gal-specific agglutinins

| | | | | |
|---|---|---|---|---|
| IRA | Iris hybrid | Iris | Monocot, | Van Damme et |
| | | | Iridaceae | al. (1998) |
| IRA b | Iris hybrid | Iris | Monocot, | Van Damme et |
| | | | Iridaceae | al. (1998) |
| IRA r | Iris hybrid | Iris | Monocot, | Van Damme et |
| | | | Iridaceae | al. (1998) |

Man/GalNAc-specific agglutinins

| | | | | |
|---|---|---|---|---|
| TL C II | Tulipa hybrid | Tulip | Monocot, | Van Damme et |
| | | | Liliaceae | al. (1998) |

In the Table 2, hereinabove, Monocot: Monocotyledoneae, dicot: Dicotyledoneae, Man: mannose, GlcNac: N-acetyl glucosamine, GalNAc: N-acetyl galactosamine, Gal: galactose, Neu5Ac: N-acetylneuraminic acid.

In the Table 2, hereinabove, Van Damme et al. (1998) refers to Van Damme, E., Peumans, W., Pusztai, A., Bardocz, S., 1998. Handbook of Plant Lectins: Properties and Biomedical Applications. John Wiley & Sons, Chichester, West Sussex, England.

In the Table 2, hereinabove, Van Damme et al. (2002) refers to Van Damme, E. J., Hause, B., Hu, J., Barre, A., Rouge, P., Proost, P., Peumans, W. J., 2002. Two distinct jacalin-related lectins with a different specificity and subcellular location are major vegetative storage proteins in the bark of the black mulberry tree. Plant Physiol. 130, 757-769.

In the Table 2, hereinabove, Chen et al. (2002) refers to Chen, Y., Peumans, W. J., Hause, B., Bras, J., Kumar, M., Proost, P., Barre, A., Rouge, P., Van Damme, E. J., 2002. Jasmonic acid methyl ester induces the synthesis of a cytoplasmic/nuclear chito-oligosaccharide binding lectin in tobacco leaves. FASEB J. 16, 905-907.

In the Table 2, hereinabove, Wang et al. (2003) refers to Wang, W., Peumans, W. J., Rouge, P., Rossi, C., Proost, P., Chen, J., Van Damme, E. J., 2003. Leaves of the Lamiaceae species Glechoma hederacea (ground ivy) contain a lectin that is structurally and evolutionary related to the legume lectins. Plant J. 33, 293-304.

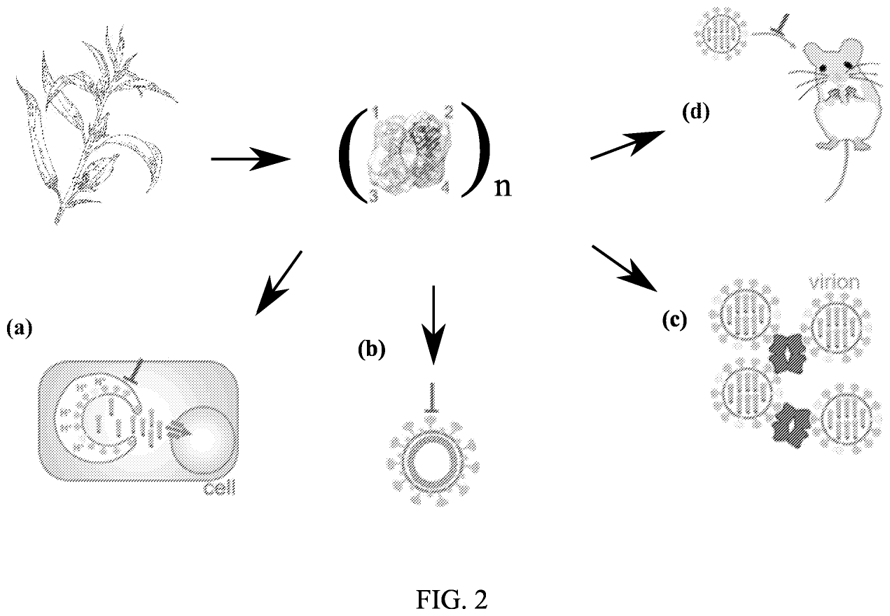

Next, the prepared recombinant polynucleotide encoding a combination of lectins as aforesaid will be introduced into crops such as corn and wheat to create transgenic crops that can help an animal or human consuming said crops gain partial or complete protection from and/or treat the effects of exposure to viral pathogens. Briefly, to create such a transgenic crop variety, known and routinely used methods which are well-known to an ordinary person of skill in the art, where the aforementioned prepared recombinant polynucleotide encoding a combination of lectins will be inserted it into the cells of a crop (for example, corn or wheat plant) at the embryo stage. The resulting mature plant will be tested to have the combination of lectins in all its cells and expression of said lectins will be tested in the leaves of the transformed plant. One such mechanism known to a person of skill in the art is schematically represented in FIG. 1(b). The test assay will involve inhibition of viral transcription, as based in established literature by the combination of lectins. The various mechanisms that are known or suggested in the art underlying the antiviral activities of lectins is schematically represented in FIG. 2. The resulting seeds from the transgenic corn and wheat plant will have anti-viral properties against viral pathogens such as the COVID-19 causing virus, SARS-CoV-2, due to the specific transcribed proteins, which will be tested in this experiment to obtain the desired transgenic plants and transgenic plant seed from the so obtained transgenic crops.

### Example 2

Microneutralization and plaque reduction assays: In this example, the 50% infective dose (TCID₅₀) and immunoplaque assay (PFU/ml) of viruses in MDCK or Vero E6 cells will be determined in this example. A protocol for the serological diagnosis of influenza by MN assay was used, with a combination of the selected lectins that is the experimental combination of lectins as is to be used to produce transgenic plants and transgenic plant seed in Example 1, along with individual lectins in the said combination for comparison, and control proteins will be used in place of sera (World Health Organization, 2011). The experimental combination of lectins, reference individual lectins for comparison, control proteins, and viruses will be incubated at 37° C. for 1 hour in a 96 well tissue culture plate, then 1.5×10⁴ cells/well will be added to the mixture. The plate will then be cultured in serum-free medium for 18˜20 hours, then washed and fixed with 50% methanol 50% acetone. Anti-NP (influenza virus) or anti-N (coronavirus) ELISA will then be used to determine virus titer. Plates will be blocked with 5% skim milk, 0.5% BSA, and rabbit polyclonal anti-NP or mouse polyclonal anti-N primary antibody and HRP-conjugated secondary antibody will be sequentially added. Peroxidase substrate solution (TMB) and 1M H₂SO₄ will be used as stop solution and the absorbance (OD 450 nm) will be read by a microplate reader (Victor3. Perkin Elmer, Waltham, Massachusetts).

Next, for the plaque reduction assay, MDCK or Vero E6 cells will be plated onto a 6-well plate at 2×10⁵ cells/well overnight for 90% confluence. The experimental combination of lectins, individual lectins in the said combination for comparison and control proteins will be co-incubated with viruses at 37° C. for 1 hour, before the mixture will be added onto the monolayer for another hour. The mixtures comprising virus/combination of lectins, virus/individual lectins in the said combination for comparison, and virus/control proteins will be aspirated, the cells washed with PBS, and a 0.5% low-melting agarose in serum-free media will be layered onto the cells. The plates will be allowed to solidify at room temperature for 30 minutes, then incubated at 37° C. for 4˜5 days or until cytopathic effects (CPE) will be observed. Afterward, cells will be fixed with 7.4% formalin, 1% tween 20, and agarose plugs will be removed. For influenza virus, immunoplaque assay will be performed with rabbit polyclonal anti-nucleoprotein (NP) primary antibody and HRP-conjugated secondary antibody, and plaques will be visualized by incubating with KPL TruBlue peroxidase substrate (Seracare, Milford, Massachusetts) overnight. For coronavirus, the plate will be stained with 0.5% crystal violet.

### Example 3

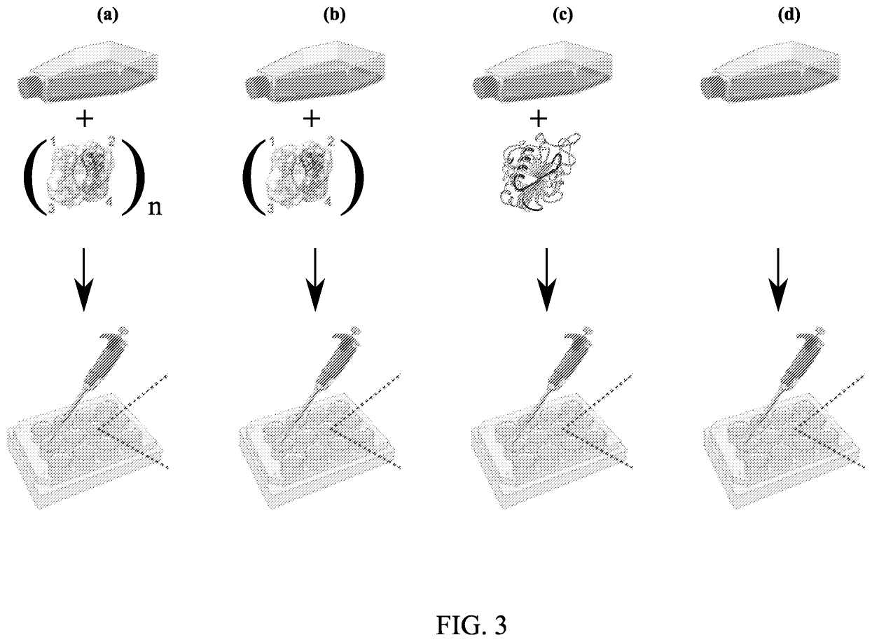

Toxicity and cell viability assays: In this example, Vero E6 cells will be used to assess in an in vitro toxicity and cell survival assay as shown in FIG. 3, where the said cells will be exposed to desired experimental concentrations of: as shown in FIG. 3(a) the experimental combination of lectins of the present disclosure as compared with as shown in FIG. 3(b) the individual lectins forming the said combination as compared with as shown in FIG. 3(c) a control protein not known to provide any protection against viral pathogens and as shown in FIG. 3(d) non-treated Vero E6 cells to experimentally assay and determine toxicity and tolerance from such exposure and treatment, where the said combination of proteins or individual proteins may either be sourced directly from the original plant sources and combined in the desired concentrations in line with the concentrations of the same extracted from experimentally produced transgenic plants and transgenic plant seed in Example 1 as disclosed herein. The said example will be used to determine and prove the high tolerance and lack of toxicity of the individual as well as combination of lectins, particularly suggesting a combination is just as well tolerated as individual lectins forming part of the experimental combination of lectins. This experiment will also act as a screen for the probable lectin candidates as shown in Table 2 and Example 1 which are toxic and will be eliminated from further experimentation.

### Example 4

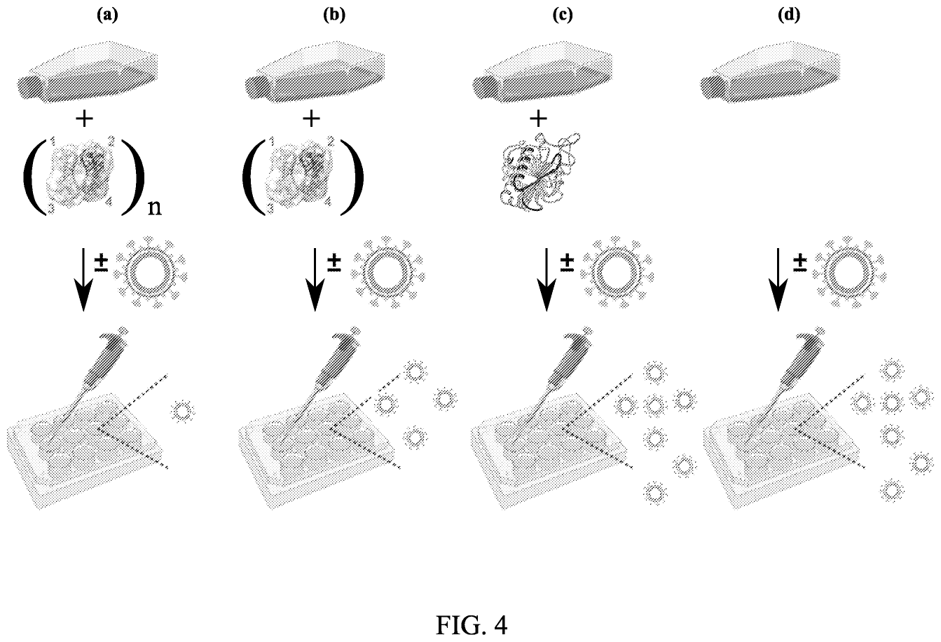

In vitro antiviral activity of experimental combination of lectins against SARS-CoV-2 infection: In this example, Vero E6 cells will be used to assess in an in vitro assay to determine the antiviral activity as shown in FIG. 4, where the said cells will be exposed to desired experimental concentrations of: as shown in FIG. 4(a) the experimental combination of lectins as compared with as shown in FIG. 4(b) the individual lectins forming the said combination as compared with as shown in FIG. 4(c) a control protein not known to provide any protection against viral pathogens and as shown in FIG. 4(d) non-treated Vero E6 cells to experimentally assay and determine protection from viral pathogens such as influenza viruses, SARS-CoV-2, feline infectious peritonitis infection in Vero E6 cells, where the said combination of proteins or individual proteins may either be sourced directly from the original plant sources and combined in the desired concentrations in line with the concentrations of the same extracted from experimentally produced transgenic plants and transgenic plant seed in Example 1 as disclosed herein. The said example will be used to determine and prove the antiviral activity of the combination of lectins is more potent will lower toxicity at the lower concentrations and yet more effective as compared with the individual lectins forming part of the experimental combination of lectins showing technical advancement and advantageous results of the present disclosure. This experiment will also act as a screen for the unsuccessful lectin candidates or ones which show decline in antiviral activity or inhibitory effect in combination or on their own when testing the candidates as shown in Table 2 and Example 1 which then will be eliminated from further experimentation.

### Example 5

Toxicity assay in vivo: In this example, provides a schematic representation of an in vivo toxicity and tolerance assay in mice models exposed to desired experimental concentrations of the experimental combination of lectins of the present disclosure as compared with the individual lectins forming the said combination as compared with a control protein not known to provide any protection against viral pathogens as well as non-treated mice to experimentally assay and determine toxicity and tolerance from such exposure and treatment, where the said combination of proteins or individual proteins may either be sourced directly from the original plant sources and combined in the desired concentrations in line with the concentrations of the same extracted from experimentally produced transgenic plants and transgenic plant seed in Example 1 as disclosed herein. The candidates selected from Example 3 and Example 4 consolidated results will be given preference.

### Example 6

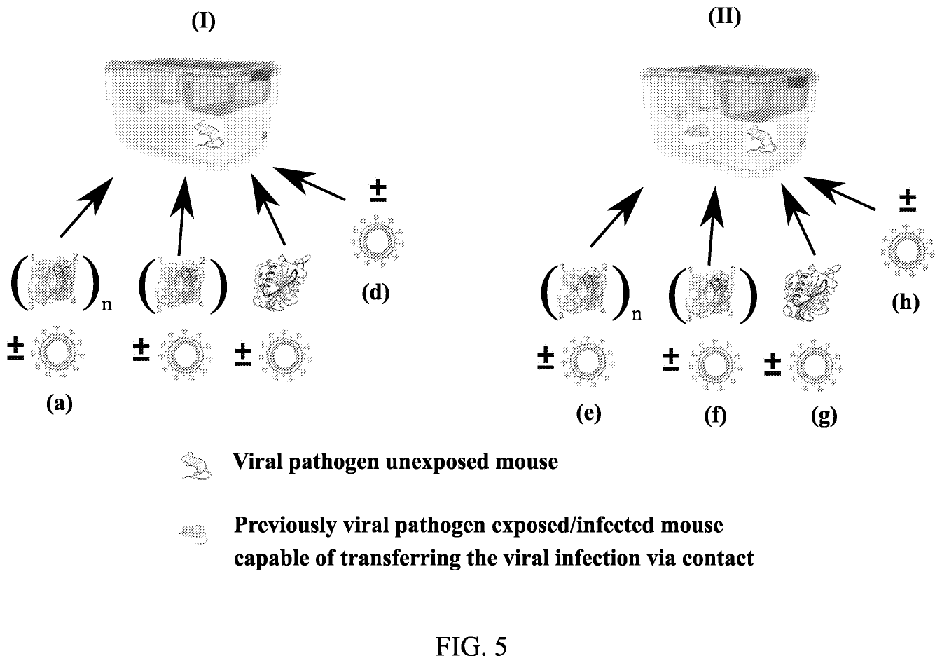

In vivo antiviral activity of experimental combination of lectins against SARS-CoV-2 infection shown for direct or contact-based infection models: In this example, as shown in FIG. 5, an in vivo antiviral activity of mice fed food pellets comprising foodstuff comprising desired experimental concentrations of (a) and (e) the experimental combination of lectins of the present disclosure as compared with (b) and (f) the individual lectins forming the said combination as compared with (c) and (g) a control protein not known to provide any protection against viral pathogens as well as (d) and (h) non-treated mice to experimentally assay and determine antiviral activity with such treatment and determine protection from viral pathogens and infections caused by pathogens such as influenza viruses, SARS-CoV-2, feline infectious peritonitis infection for mouse models identified as (I) direct exposure to viral pathogen based infection, and (II) contact-based infection transfer from an already infected mouse to unexposed mouse, where the said combination of proteins or individual proteins may either be sourced directly from the original plant sources and combined in the desired concentrations in line with the concentrations of the same extracted from experimentally produced transgenic plants and transgenic plant seed in Example 1 as disclosed herein. The candidates selected from Example 3 and Example 4 consolidated results will be given preference.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. A transgenic plant seed for inhibiting viral replication and providing immunity against viral pathogens, wherein the genome of said seed comprises an exogenous recombinant polynucleotide encoding a combination of polypeptides, wherein said polypeptides are carbohydrate binding proteins, and wherein said seed exhibit inhibition of viral replication and provide immunity against viral pathogens.

2. The transgenic plant seed of claim 1, wherein the polypeptides in the combination of polypeptides are carbohydrate binding proteins, and wherein the combination of polypeptides is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides.

3. The transgenic plant seed of claim 1, wherein the combination of polypeptides is a combination of carbohydrate binding proteins which are encoded by the exogenous recombinant polynucleotide encoding the combination of polypeptides, wherein the exogenous recombinant polynucleotide encoding the combination of polypeptides is under the control of promoters which are selected from heterologous and homologous promoters, wherein the promoters lead to ubiquitous expression or a plant part specific targeted expression from the exogenous recombinant polynucleotide encoding the combination of polypeptides, and wherein the promoters are functional in a plant cell and operably joined to encoding sequence of the recombinant polynucleotide encoding the combination of polypeptides.

4. The transgenic plant seed of claim 1, wherein the combination of polypeptides is a combination of carbohydrate binding proteins, wherein the carbohydrate binding proteins are combined in a manner to overcome potential toxicity caused by transgenic expression of the carbohydrate binding proteins in the transgenic plant seed or plants grown from said seed and to lower the concentrations of each of the carbohydrate binding proteins when expressed as a part of the combination of carbohydrate binding proteins in the combination when compared with transgenic expression of any one of the carbohydrate binding proteins in the transgenic plant seed or plants grown from said seed individually, and wherein the concentration of the each of the carbohydrate binding proteins in the combination is in an approximate range of between 1 milligram/milliliter or 1 mg/ml to 1 attogram/milliliter or 1 ag/ml, usually in a typical range of between 50 microgram/milliliter or 50 μg/ml to 1 picogram/milliliter or 1 pg/ml. Some extraordinarily potent proteins and/or peptide sequences may have activity down to 1-10 ag/mL or 1-10 attogram/mL (attomolar concentration).

5. The transgenic plant seed of claim 1, wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

6. The transgenic plant seed of claim 1, wherein the carbohydrate binding proteins are lectins obtained from a plurality of sources that comprise glucose/mannose lectin or Dolichos lablab lectin 1 (DLL-I) from Lablab purpureus or lablab beans, GlcNAc-specific agglutinins from Nicotiana sp. or tobacco, mannose-specific agglutinin from Allium sp. or leek, mannose-specific agglutinin from Galanthus sp., mannose-specific lectin from rhizomes of Ophiopogon japonicus, chitin-specific lectin from rhizome of Setcreasea purpurea, Serpula vermicularis lectin (SVL), mannose-specific agglutinin from Hippeastrum hybrid or amaryllis, mannose-specific agglutinin from Galanthus nivalis or snowdrop, mannose-specific agglutinin from Narcissus pseudonarcissus or daffodil, mannose-specific agglutinin from Lycoris radiata or red spider lily, mannose-specific agglutinin from Allium porrum or leek, mannose-specific agglutinin from Allium ursinum or ramsons, mannose-specific agglutinin from Allium sativum or garlic, mannose-specific agglutinin from Colocasia esculenta or taro, mannose-specific agglutinin from Cymbidium hybrid or Cymbidium orchid, mannose-specific agglutinin from Listera ovata or twayblade, mannose-specific agglutinin from Epipactis helleborine or broad-leaved helleborine, mannose-specific agglutinin from Tulipa hybrid or tulip, mannose-specific agglutinin from Morus nigra or black mulberry tree, GlcNAc-specific agglutinins from Phragmites australis or common reed, GlcNAc-specific agglutinins from Nicotiana tabacum or tobacco plant, (GlcNAc)ₙ-specific agglutinin from Urtica dioica or stinging nettle, (GlcNAc)ₙ-specific agglutinin from Hevea brasiliensis or rubber tree, GalNAc-specific agglutinin from Polygonatum multiflorum tetramer or solomon's seal, GalNAc-specific agglutinin from Bryonia dioica or white bryony, GalNAc-specific agglutinin from Glechoma hederacea or ground ivy, Gal-specific agglutinin from Morus nigra or black mulberry tree, Gal-specific agglutinin from Artocarpus integrifolia or jackfruit, Neu5Acα(2.6)Gal/GalNAc-specific agglutinin from Sambucus nigra or elderberry, Man/Glc-specific agglutinin from Cladastris lutea or yellow wood, Gal/GalNAc-specific agglutinin from Polygonatum multiflorum monomer or solomon's Seal, Gal/GalNAc-specific agglutinin from Viscum album or mistletoe, GalNAc (>Gal) specific agglutinin from Viscum album or mistletoe, GalNAcα(1,3)Gal>GalNAc>Gal-specific agglutinin from Iris hybrid or Iris, Man/GalNAc-specific agglutinin from Tulipa hybrid or tulip, Hippeastrum hybrid agglutinin (HHA), Galanthus nivalis agglutinin (GNA), Cymbidium sp. agglutinin (CA), Urtica dioica agglutinin (UDA), Scytovirin (SVN) isolated from cyanobacterium Scytonema varium, carbohydrate binding proteins isolated from the sea coral Gerardia savaglia (GSA), Griffithsin derived from a red alga Griffithsia sp., and Actinohivin derived from the actinomycete Longisporum albida.

7. The transgenic plant seed of claim 1, wherein the viral pathogens comprise coronaviruses, rhabdoviruses, influenza viruses, dengue viruses, severe acute respiratory syndrome coronaviruses (SARS-CoV), severe acute respiratory syndrome coronavirus 1 (SARS-CoV-1), severe acute respiratory syndrome coronavirus 2 (SARS-CoV-2), Middle East respiratory syndrome coronaviruses (MERS-CoV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses, HCoV-OC43, HCoV-229E, HCoV-NL63, HCoV-HKU1, feline infectious peritonitis virus (FIPV), feline enteric coronavirus (FECV), rinderpest virus, foot-and-mouth disease virus (FMDV), cypoviruses (CPV), and reoviruses.

8. The transgenic plant seed of claim 1, wherein the transgenic plant seed produces plants and plant parts that exhibit inhibition of viral replication and provide immunity against viral pathogens, wherein the plants comprise corn, wheat, millets, rye, oats, barley, sorghum, rice, legumes, nuts, and tubers.

9. The transgenic plant seed of claim 1, wherein the transgenic plant seed are sown to produce plants and plant parts that are mass produced with agricultural practices, horticulture practices, and in green house gardens, wherein said plant parts comprise grains, harvested seeds, and by-products, wherein said mass produced plants and plant parts are processed to produce foodstuff, wherein said foodstuff comprise bread, cereal, flour, baby food, snack food, pet food, dried soups, dry beverage mixes, and texturized vegetable proteins, wherein said by-products comprise bran, middlings, mill run, shorts, red dog, screenings, germ meal, and germ oil, and wherein said by-products produce animal feed and manures, and wherein said foodstuff exhibit inhibition of viral replication and provide immunity against viral pathogens.

10. A recombinant polynucleotide encoding a combination of polypeptides, wherein said polypeptides are carbohydrate binding proteins, wherein the carbohydrate binding proteins are encoded by the recombinant polynucleotide encoding the combination of polypeptides, wherein the recombinant polynucleotide encoding the combination of polypeptides is under the control of promoters which are selected from heterologous and homologous promoters, wherein the promoters lead to ubiquitous expression or a plant part specific targeted expression from the recombinant polynucleotide encoding the combination of polypeptides, wherein the promoters are functional in a plant cell and operably joined to encoding sequence of the recombinant polynucleotide encoding the combination of polypeptides, wherein the combination of polypeptides is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides, and wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

11. The recombinant polynucleotide of claim 10, wherein the carbohydrate binding proteins are lectins obtained from a plurality of sources that comprise glucose/mannose lectin or Dolichos lablab lectin 1 (DLL-I) from Lablab purpureus or lablab beans, GlcNAc-specific agglutinins from Nicotiana sp. or tobacco, mannose-specific agglutinin from Allium sp. or leek, mannose-specific agglutinin from Galanthus sp., mannose-specific lectin from rhizomes of Ophiopogon japonicus, chitin-specific lectin from rhizome of Setcreasea purpurea, Serpula vermicularis lectin (SVL), mannose-specific agglutinin from Hippeastrum hybrid or amaryllis, mannose-specific agglutinin from Galanthus nivalis or snowdrop, mannose-specific agglutinin from Narcissus pseudonarcissus or daffodil, mannose-specific agglutinin from Lycoris radiata or red spider lily, mannose-specific agglutinin from Allium porrum or leek, mannose-specific agglutinin from Allium ursinum or ramsons, mannose-specific agglutinin from Allium sativum or garlic, mannose-specific agglutinin from Colocasia esculenta or taro, mannose-specific agglutinin from Cymbidium hybrid or Cymbidium orchid, mannose-specific agglutinin from Listera ovata or twayblade, mannose-specific agglutinin from Epipactis helleborine or broad-leaved helleborine, mannose-specific agglutinin from Tulipa hybrid or tulip, mannose-specific agglutinin from Morus nigra or black mulberry tree, GlcNAc-specific agglutinins from Phragmites australis or common reed, GlcNAc-specific agglutinins from Nicotiana tabacum or tobacco plant, (GlcNAc)ₙ-specific agglutinin from Urtica dioica or stinging nettle, (GlcNAc)ₙ-specific agglutinin from Hevea brasiliensis or rubber tree, GalNAc-specific agglutinin from Polygonatum multiflorum tetramer or solomon's seal, GalNAc-specific agglutinin from Bryonia dioica or white bryony, GalNAc-specific agglutinin from Glechoma hederacea or ground ivy, Gal-specific agglutinin from Morus nigra or black mulberry tree, Gal-specific agglutinin from Artocarpus integrifolia or jackfruit, Neu5Acα(2.6)Gal/GalNAc-specific agglutinin from Sambucus nigra or elderberry, Man/Glc-specific agglutinin from Cladastris lutea or yellow wood, Gal/GalNAc-specific agglutinin from Polygonatum multiflorum monomer or solomon's Seal, Gal/GalNAc-specific agglutinin from Viscum album or mistletoe, GalNAc (>Gal) specific agglutinin from Viscum album or mistletoe, GalNAcα(1,3)Gal>GalNAc>Gal-specific agglutinin from Iris hybrid or Iris, Man/GalNAc-specific agglutinin from Tulipa hybrid or tulip, Hippeastrum hybrid agglutinin (HHA), Galanthus nivalis agglutinin (GNA), Cymbidium sp. agglutinin (CA), Urtica dioica agglutinin (UDA), Scytovirin (SVN) isolated from cyanobacterium Scytonema varium, carbohydrate binding proteins isolated from the sea coral Gerardia savaglia (GSA), Griffithsin derived from a red alga Griffithsia sp., and Actinohivin derived from the actinomycete Longisporum albida.

12. A method for treating or preventing a viral disease, comprising administering to an individual in need an effective amount of a foodstuff derived from plants and plant parts obtained from a transgenic plant seed, the method comprising the steps of:
(a) making a recombinant polynucleotide encoding a combination of polypeptides, wherein said polypeptides are carbohydrate binding proteins, wherein the carbohydrate binding proteins are encoded by the recombinant polynucleotide encoding the combination of polypeptides, wherein the recombinant polynucleotide encoding the combination of polypeptides is under the control of promoters which are selected from heterologous and homologous promoters, wherein the promoters lead to ubiquitous expression or a plant part specific targeted expression from the recombinant polynucleotide encoding the combination of polypeptides, wherein the promoters are functional in a plant cell and operably joined to encoding sequence of the recombinant polynucleotide encoding the combination of polypeptides;
(b) transforming plant cells by delivering the recombinant polynucleotide of step (a) and regenerating full fertile transformed plants from said cells in vitro, wherein the transformed plants express combination of polypeptides, wherein said polypeptides are carbohydrate binding proteins;
(c) growing said transformed plants to obtain a transgenic plant seed;
(d) sowing said transgenic plant seed to produce transgenic plants and plant parts that are mass produced with agricultural practices, horticulture practices, and in green house gardens; and
(e) processing said transgenic plants and plant parts to obtain foodstuff, wherein said foodstuff exhibit inhibition of viral replication and provide immunity against viral pathogens, wherein said plant parts comprise grains, harvested seeds, and by-products, wherein said mass produced plants and plant parts are processed to produce foodstuff, wherein said foodstuff comprise bread, cereal, flour, baby food, snack food, pet food, dried soups, dry beverage mixes, and texturized vegetable proteins, wherein said by-products comprise bran, middlings, mill run, shorts, red dog, screenings, germ meal, and germ oil, wherein said by-products produce animal feed and manures, and wherein the administering to an individual in need an effective amount of a foodstuff leads to reduction or elimination of viral reservoirs in the individual, wherein said viral reservoirs include viral deposits in gut and central nervous system (CNS) of the individual.

13. The method for treating or preventing a viral disease of claim 12, wherein the combination of polypeptides is a combination of carbohydrate binding proteins in step (a) and is selected from a group comprising a combination of two different carbohydrate binding proteins for expression from the exogenous recombinant polynucleotide encoding the combination of polypeptides, three different carbohydrate binding proteins for expression from the exogenous recombinant polynucleotide encoding the combination of polypeptides, four different carbohydrate binding proteins for expression from the exogenous recombinant polynucleotide encoding the combination of polypeptides, and five different carbohydrate binding proteins for expression from the exogenous recombinant polynucleotide encoding the combination of polypeptides.

14. The method for treating or preventing a viral disease of claim 12, wherein the combination of polypeptides is a combination of carbohydrate binding proteins in step (a) and the carbohydrate binding proteins are combined in a manner to overcome potential toxicity caused by transgenic expression of the carbohydrate binding proteins in the transgenic plant seed or plants grown from said seed and to lower the concentrations of each of the carbohydrate binding proteins when expressed as a part of the combination of carbohydrate binding proteins in the combination when compared with transgenic expression of any one of the carbohydrate binding proteins in the transgenic plant seed or plants grown from said seed individually, and wherein the concentration of the each of the carbohydrate binding proteins in the combination is in an approximate range of between 1 milligram/milliliter or 1 mg/ml (millimolar) to 1 attogram/milliliter or 1 ag/ml (attomolar), usually in a typical range of between 50 microgram/milliliter or 50 μg/ml to 1 picogram/milliliter or 1 pg/ml. Some extraordinarily potent proteins and/or peptide sequences may have activity down to 1-10 ag/mL or 1-10 attogram/mL (attomolar concentration).

15. The method for treating or preventing a viral disease of claim 12, wherein the carbohydrate binding proteins comprise mannose-specific plant lectins, N-acetyl glucosamine-specific plant lectins, glucose-specific plant lectins, galactose-specific plant lectins, N-acetyl galactosamine-specific plant lectins, galactose-specific plant agglutinins, N-acetylgalactosamine-specific plant agglutinins, glucose-specific plant agglutinins, and N-acetylglucosamine-specific plant agglutinins.

16. The method for treating or preventing a viral disease of claim 12, wherein the carbohydrate binding proteins are lectins obtained from a plurality of sources that comprise glucose/mannose lectin or Dolichos lablab lectin 1 (DLL-I) from Lablab purpureus or lablab beans, GlcNAc-specific agglutinins from Nicotiana sp. or tobacco, mannose-specific agglutinin from Allium sp. or leek, mannose-specific agglutinin from Galanthus sp., mannose-specific lectin from rhizomes of Ophiopogon japonicus, chitin-specific lectin from rhizome of Setcreasea purpurea, Serpula vermicularis lectin (SVL), mannose-specific agglutinin from Hippeastrum hybrid or amaryllis, mannose-specific agglutinin from Galanthus nivalis or snowdrop, mannose-specific agglutinin from Narcissus pseudonarcissus or daffodil, mannose-specific agglutinin from Lycoris radiata or red spider lily, mannose-specific agglutinin from Allium porrum or leek, mannose-specific agglutinin from Allium ursinum or ramsons, mannose-specific agglutinin from Allium sativum or garlic, mannose-specific agglutinin from Colocasia esculenta or taro, mannose-specific agglutinin from Cymbidium hybrid or Cymbidium orchid, mannose-specific agglutinin from Listera ovata or twayblade, mannose-specific agglutinin from Epipactis helleborine or broad-leaved helleborine, mannose-specific agglutinin from Tulipa hybrid or tulip, mannose-specific agglutinin from Morus nigra or black mulberry tree, GlcNAc-specific agglutinins from Phragmites australis or common reed, GlcNAc-specific agglutinins from Nicotiana tabacum or tobacco plant, (GlcNAc)ₙ-specific agglutinin from Urtica dioica or stinging nettle, (GlcNAc)ₙ-specific agglutinin from Hevea brasiliensis or rubber tree, GalNAc-specific agglutinin from Polygonatum multiflorum tetramer or solomon's seal, GalNAc-specific agglutinin from Bryonia dioica or white bryony, GalNAc-specific agglutinin from Glechoma hederacea or ground ivy, Gal-specific agglutinin from Morus nigra or black mulberry tree, Gal-specific agglutinin from Artocarpus integrifolia or jackfruit, Neu5Acα(2.6)Gal/GalNAc-specific agglutinin from Sambucus nigra or elderberry, Man/Glc-specific agglutinin from Cladastris lutea or yellow wood, Gal/GalNAc-specific agglutinin from Polygonatum multiflorum monomer or solomon's Seal, Gal/GalNAc-specific agglutinin from Viscum album or mistletoe, GalNAc (>Gal) specific agglutinin from Viscum album or mistletoe, GalNAcα(1,3)Gal>GalNAc>Gal-specific agglutinin from Iris hybrid or Iris, Man/GalNAc-specific agglutinin from Tulipa hybrid or tulip, Hippeastrum hybrid agglutinin (HHA), Galanthus nivalis agglutinin (GNA), Cymbidium sp. agglutinin (CA), Urtica dioica agglutinin (UDA), Scytovirin (SVN) isolated from cyanobacterium Scytonema varium, carbohydrate binding proteins isolated from the sea coral Gerardia savaglia (GSA), Griffithsin derived from a red alga Griffithsia sp., and Actinohivin derived from the actinomycete Longisporum albida.

17. The method for treating or preventing a viral disease of claim 12, wherein the viral pathogens comprise coronaviruses, rhabdoviruses, influenza viruses, dengue viruses, severe acute respiratory syndrome coronaviruses (SARS-CoV), severe acute respiratory syndrome coronavirus 1 (SARS-CoV-1), severe acute respiratory syndrome coronavirus 2 (SARS-CoV-2), Middle East respiratory syndrome coronaviruses (MERS-CoV), Orthomyxoviruses, hepatitis viruses, hepatitis C virus (HCV), hepatitis E virus (HEV), ebola viruses, polio measles viruses, retroviruses, adult human T-cell lymphotropic virus type 1 (HTLV-1), human immunodeficiency viruses (HIV), noroviruses, common cold viruses, west nile fever virus, rabies viruses, polio viruses, mumps viruses, measles viruses, chikungunya viruses, zika viruses, herpes simplex viruses, HCoV-OC43, HCoV-229E, HCoV-NL63, HCoV-HKU1, feline infectious peritonitis virus (FIPV), feline enteric coronavirus (FECV), rinderpest virus, foot-and-mouth disease virus (FMDV), cypoviruses (CPV), and reoviruses.

18. The method for treating or preventing a viral disease of claim 12, wherein the transformed plants in step (c) and consequent transgenic plants in step (e) comprise corn, wheat, millets, rye, oats, barley, sorghum, rice, legumes, nuts, and tubers.

19. The method for treating or preventing a viral disease of claim 12, wherein the effective amount of a foodstuff derived from plants and plant parts obtained from a transgenic plant seed is administered along with one or more antivirals, wherein antivirals comprise Remdesivir, Nirmatrelvir with Ritonavir (Paxlovid), and Molnupiravir.

20. The method for treating or preventing a viral disease of claim 12, wherein the individual comprises human beings, domesticated animals, farm animals, zoo animals, agricultural beasts, and wild animals.
